(19) United States
(12) Patent Application Publication
(10) Pub. No.: US 2013/0317107 A1
(43) Pub. Date: Nov. 28, 2013

(54) Platelet-Based Methods to Detect and Monitor Treatment Benefits in Mucosal and Nervous Systems Diseases

(71) Applicants: Leonore A. Herzenberg, Stanford, CA (US); Rabin Tirouvanziam, Redwood City, CA (US); Tetyana Obukhanyck, Palo Alto, CA (US); Yael Gernerz, San Francisco, CA (US)

(72) Inventors: Leonore A. Herzenberg, Stanford, CA (US); Rabin Tirouvanziam, Redwood City, CA (US); Tetyana Obukhanyck, Palo Alto, CA (US); Yael Gernerz, San Francisco, CA (US)

(21) Appl. No.: 13/800,051

(22) Filed: Mar. 13, 2013

Related U.S. Application Data
(60) Provisional application No. 61/688,947, filed on May 25, 2012.

(51) Int. Cl. G01N 33/86; A61K 31/198

(52) U.S. Cl. CPC G01N 33/86; A61K 31/198
USPC 514/562; 435/7.92; 506/9

(57) ABSTRACT

The described invention provides methods for treating a disease, disorder or condition comprising an inflammatory component that includes a platelet dysfunction component and methods for monitoring therapeutic efficacy of a therapeutic regimen for managing a disease comprising an inflammatory component that includes platelet dysfunction.

### CROSS REFERENCE TO RELATED APPLICATIONS

This application claims the benefit of priority to U.S. Provisional Application 61/668,947 (filed Jul. 6, 2012), and is a continuation-in-part of U.S. application Ser. No. 11/507,706, entitled “Methods for Treating and Monitoring Inflammation and Redox Imbalance Cystic Fibrosis,” filed Aug. 22, 2006, which claims the benefit of priority from U.S. Provisional Applications No. 60/710,807 filed Aug. 24, 2005. Each of these applications is incorporated by reference herein in its entirety.

### STATEMENT OF GOVERNMENT FUNDING

The described invention was made with government support. The government has certain rights in the invention.

### FIELD OF INVENTION

The described invention is related to methods for treating a disease, disorder or condition comprising an inflammatory component that includes a platelet dysfunction component and methods for monitoring therapeutic efficacy of a therapeutic regimen for managing such diseases, disorders or conditions.

### BACKGROUND

### 1. The Platelet

Platelets are small fragments of megakaryocyte cytoplasm having an average lifespan in the peripheral circulation of 1-10 days. Their function is to maintain the integrity of the vessel wall and to initiate hemostasis upon damage to the vasculature. Their functionality can be divided into three main areas: adhesion to the vascular endothelium; aggregation to each other; and release of chemicals into the plasma (Burnett, D. et al., 2005, The Science of Laboratory Diagnosis, John Wiley and Sons, pp. 289-293).

Platelets circulate around the body in an inactive state until they come into contact with areas of endothelial damage or activation of the coagulation cascade where they adhere to the endothelial defect, change shape, release their granule contents, and stick together to form aggregates. Physiologically, these processes help to limit blood loss; however, inappropriate or excessive platelet activation results in an acute obstruction of blood flow, as occurs, for example, in an acute myocardial infarction. However, activated platelets also express and release molecules that stimulate a localized inflammatory response through the activation of leukocytes and endothelial cells. Accumulating evidence suggests that platelet function is not merely limited to the prevention of blood loss. For example, platelets have been implicated in many pathological processes including host defense, inflammatory arthritis, adult respiratory distress syndrome, and tumor growth and metastasis (Quinn M. et al., 2005, Platelet Function: assessment, diagnosis, and treatment, Humana Press, pp. 3-20).

Platelet function is related closely to the platelet structure. The platelet external membrane is a highly functional organelle, containing different glycoproteins that transect the standard lipid bilayer cellular membrane. Several of these glycoproteins are capable of activating various biochemical pathways within the platelet to induce the functions required in a normal platelet response. Abnormal platelet function has been shown to result from a lack of expression of one or more of these glycoproteins, including, GP IIb-IIIa, GP Ia-IIa, and GMP 140 (platelet activation dependent granule-external membrane protein (PADGEM)). The glycoproteins act as a physiological receptor for various low and high molecular weight platelet agonists. Once one or more of these glycoprotein receptors have become activated, there is a signal transduction into the internal organelles of the platelet. This is commonly via activation of the enzyme phospholipase C, which is exposed on the internal ends of the glycoproteins upon activation by their respective ligands (Burnett, D. et al., 2005, The Science of Laboratory Diagnosis, John Wiley and Sons, pp. 289-293).

Within the platelet cytoplasm are many of the internal structures found in other secretory cells; however, the platelet does not have a great capacity for the synthesis of proteins. The platelet contains very little rough endoplasmic reticulum and Golgi apparatus, but does contain extensive smooth endoplasmic reticulum, which is often referred to as “the dense tubular system.” The cytoplasm also contains any alphα-granules and dense granules. These granules contain a wide variety of chemicals, which are involved in the inflammatory response and in accelerating the process of localized hemostasis. The alphα-granules contain coagulation factors such as factors V, VII and fibrinogen, along with growth factors to aid vascular repair, notably platelet-derived growth factor (PDGF) and endothelial growth factor (EGF). Another substance stored in alphα-granules is platelet factor IV, and this is one of the main factors assayed to assess the platelet release response (Burnett, D. et al., 2005, The Science of Laboratory Diagnosis, John Wiley and Sons, pp. 289-293).

Platelets possess an anatomic and biochemical machinery in many aspects comparable to leukocytes and a series of structural characteristics that may be relevant to inflammation. Moreover, platelets are known to inherit several characteristics from their bone marrow progenitor cells, megakaryocytes, and these are provided with vivacious cell locomotion. (Gresele, P. et al., 2002, Platelets in thrombotic and non-thombotic disorders: pathophysiology, pharmacology and therapeutics, Cambridge University Press, pp. 392-411).

Regarding the first step in cellular transmigration (i.e., adhesion to the endothelial monolayer), platelets possess and/or express a number of adhesive proteins or adhesive protein counterreceptors. Platelets contain P-selectin stored in α-granules, and express its ligand PSGL-1 on their surface; although platelets can express P-selectin on their surface upon activation, this does not regulate platelet rolling in vivo on activated endothelium while it is involved probably in the crosstalk between platelets and leukocytes. On the other hand, platelets interact with both P- and E-selectin exposed on activated endothelium through their constitutively expressed PSGL-1 receptor. Platelets have been observed while rolling in vivo on an activated endothelial surface in a manner, which is similar to that observed for leukocytes: both cell types roll on stimulated vessel wall and for both this process is dependent on the expression of endothelial P-selectin (Gresele, P. et al., 2002, Platelets in thrombotic and non-thombotic disorders: pathophysiology, pharmacology and therapeutics, Cambridge University Press, pp. 392-411).

Regarding the intracellular changes required to express cell locomotion, platelets have a cytoskeletal framework that allows cell motion. In resting platelets, the discoid shape is maintained by a network of actin filaments, spectrin, and integrins that together form the membrane skeleton, a submembraneous structure that coats internally the cytoplasmic surface of platelets, and by actin gel filaments that link this structure to transmembrane proteins. After stimulation, the activation of low molecular weight G proteins, such as Rac-1, induces the formation of focal complexes, which are very dynamic structures that are replaced by focal adhesions as the cells spread. Additional cytoskeletal changes lead to the formation of stress fibers, under the induction of RhoA, a member of the Rho family of low molecular weight GTPases. Stress fibers associate with focal adhesions allowing a contractile response to be exerted on the extracellular integrin-associated ligands. The continuous formation of filopodia and lamellipodia that leads to focal complexes is due to actin dynamic polymerization. One of the signaling molecule that is activated as consequence of integrin-induced signals is calpain. Platelets contain in their cytosol μ-calpain (one of the two major forms) that becomes activated when platelets aggregate in response to stimuli or when they spread on extracellular matrix proteins. Calpain, by inducing Rac-1 and RhoA activation, provokes integrin-induced formation of focal adhesions and actin filament reorganization (Gresele, P. et al., 2002, Platelets in thrombotic and non-thombotic disorders: pathophysiology, pharmacology and therapeutics, Cambridge University Press, pp. 392-411).

Concerning the soluble stimuli that control migration, recent reports have shown the surface expression of different chemokine receptors on platelets. The receptors for chemokines are seven transmembrane domain structures linked to G-proteins that mediate calcium flux upon activation. Recently, by using flow cytometry, immunoprecipitation, western blotting, and reverse transcriptase polymerase chain reaction, it was shown that human platelets express CCR1, CCR3, CCR4, and CXCR4 chemokine receptors. The effect of various cytokines and chemokines (e.g., Interleukin-8 (IL-8), Monocyte Chemotactic Protein-3 (MCP-3), Monocyte Chemotactic Protein-1 (MCP-1), Macrophage Inflammatory Protein-1 (MIP-1α/CCL3), eotaxin, Regulated upon Activation, Normal T cell Expressed and Secreted (RANTES/CCL-5), Thymus Activation-Regulated Chemokine (TARC), Macrophage-Derived Chemokine (MDC) and Stromal cell-Derived Factor-1 alpha (SDF-1α/CXCL12) on Ca²⁺ levels in platelets also was tested. Most of the ligands tested gave clear calcium signals. A cellular response to MIP-1α and RANTES implicates the presence of the CCR1 receptor. A response to eotaxin and RANTES implicates the presence of CCR3, while a response to TARC and MDC implicates the existence of CCR4. Finally, a response to SDF-1α implicates the expression of the CXCR4 receptor. Although the function of these chemokine receptors on platelets is not yet clear, reports have suggested that some chemokines can activate platelets (Gresele, P. et al., 2002, Platelets in thrombotic and non-thombotic disorders: pathophysiology, pharmacology and therapeutics, Cambridge University Press, pp. 392-411).

Previous studies have suggested that PF-4/CXCL4, an α-granule protein of platelets, which is also a CXC chemokine, may bind to the platelet surface and may modulate platelet aggregation and secretion induced by low levels of platelet agonists even though a specific receptor for PF4/CXCL4 has yet to be identified. Another study has reported that SDF-1α, a chemokine highly expressed in human atherosclerotic plaques, induces platelet aggregation and calcium signaling. Most recently, MDC and TARC, in addition to SDF-1α, were shown to induce platelet activation in a rapid (less than 5 seconds) and maximal way under arterial flow conditions, by facilitating the agonistic activity of low doses of primary agonists such as ADP, and their effects are insensitive to indomethacin. These data indicate that the effects of MDC and TARC are mediated by their common receptor CCR4 independent of cycloxygenase.

Platelets themselves contain some of the chemokines that are ligands for CCR1 and CCR3, such as RANTES or MIP-1α, suggesting a role for the chemokines in feeding back to receptors on the same or other platelets to amplify the response to stimuli. On the other hand, platelets do not contain either CCR4 (MDC, TARC) or CXCR4(SDF-1α) agonists. Therefore, it has been suggested that these receptors are involved in situations where these agonists are provided by other cells for platelet migration. Activated platelets have been shown to exhibit membrane-bound IL-1 bioactivity. Using immunocytological and flow cytometric techniques, IL-1α and IL-β were found in the cytoplasm of both resting and thrombin activated-platelets, and IL-1 was shown to influence indirectly the transendothelial migration of leukocytes. Moreover, flow cytometry studies have shown that the surface of platelets is able to express IL-1R and IL-8R (receptors for IL-1 and IL-8, respectively), the expression of which is significantly increased in inflammatory bowel disease (Gresele, P. et al., 2002, Platelets in thrombotic and non-thombotic disorders: pathophysiology, pharmacology and therapeutics, Cambridge University Press, pp. 392-411).

### 2. Platelet Activation

Platelet activation describes the process that converts the smooth, nonadherent platelet into a sticky spiculated particle that releases and expresses biologically active substances and acquires the ability to bind the plasma protein fibrinogen. Activation can also occur as a result of the physical stimulus of high fluid shear stress, such as that found at the site of a critical arterial narrowing (Quinn M. et al., 2005, Platelet Function: assessment, diagnosis, and treatment, Humana Press, pp. 3-20).

Normally, platelets circulate freely in blood vessels without interacting with each other or the vascular endothelium. In the context of endothelial damage, a chain of events leads to the aggregation of platelets. Depending on the nature of the vascular injury, this may develop into either a normal hemostasis or a pathologic condition (the latter resulting in vascular thrombosis, ischemic stroke, and the like). The underlying platelet events constitute a complex series of biochemical and cellular processes that can be classified as adhesion of platelets to damaged vessel wall, activation of platelets, secretion of granular contents from activated platelets, and aggregation of platelets. (Weiss, J., New Engl. J. Med., 1975, 293, 531; Weiss, J., New Engl. J. Med., 1975, 293, 580)

The adhesion of platelets to denuded endothelium represents the primary hemostatic response to vessel wall injury (Ruggeri, Z et al., Blood, 1999, 94, 172). When endothelial layer disruption occurs as a result of a vascular trauma, platelets adhere to the exposed endothelium to form a discontinuous platelet monolayer. The adhered platelets interact with sub-endothelial collagen, which causes their activation. Activation of platelets prompts cytoskeletal rearrangements, membrane fusion, exteriorization, and internal synthesis and release of thromboxane A2(TχA2), which itself is a potent platelet activator. Secretion of platelet granular contents releases a variety of biochemical agonists, such as adenosine diphosphate (ADP) and serotonin, which further activate platelets by interacting with their specific platelet surface receptors (FIG. 1) (Andrews, R. and Berndt M., Histol. Histopathol., 1998, 13, 837; Shankar, H. et al., Curr. Drug Targets, 2006, 7, 1253). Additionally, thrombin that is locally generated at the site of injury activates platelets via protease-activated receptors (PARs) (Banish, J et al., Accounts in Drug Discovery: Case Studies in Medicinal Chemistry, 2010, Royal Society of Chemistry, 2010).

Platelet activation mechanisms and activation of the coagulation system synergize in thrombus formation. Platelets have different cell-surface receptors that are activated by specific agonists (FIG. 1). Upon activation, these receptors trigger intracellular signal transduction mechanisms, resulting in the activation of integrin receptors GP IIb/IIIa. Activated GP IIb/IIIa receptors bind to the arginyl-glycyl-aspartic acid (RGD) motif of fibrinogen, thereby cross-linking activated platelets, which leads to platelet aggregation. Among the various platelet surface receptors, PAR-1 (thrombin receptor) is by far the most potent inducer of platelet activation. Concurrent with platelet activation, the coagulation process is triggered also by the exposure of tissue factor to blood, resulting in the production of thrombin. In its procoagulant role, thrombin cleaves soluble fibrinogen to fibrin, which cross-links to form an insoluble fibrin meshwork that traps aggregated platelets and other plasma particles, leading to a growing thrombus.

### 3. Platelets and Inflammation

Activated platelets play an important role in inflammation and express or release a number of molecules that lead to leukocyte activation and secretion (Gawaz M. et al., Circulation, 1998, 98:1164-1171). One of the most important immune mediators expressed by platelets is the CD40 ligand (L), a 33-kDa transmembrane protein related to TNF-α (Henn V et al., Nature, 1998, 391:591-594). Platelets are the major intravascular source of CD40L, which interacts with its receptor CD40, inducing endothelial expression of E-selectin, vascular cell adhesion molecule (VCAM)-1 and ICAM-1 and secretion of the chemokines interleukin-8 (IL-8) and monocyte chemoattractant protein (MCP-1). Platelet CD40L expression is down-regulated rapidly through cleavage and release of its less active soluble form (Henn V et al., Blood, 2001 98:1047-1054). Released CD40L binds to GPIIb-IIIa and promotes platelet aggregation under shear. In animal models of atherosclerosis, CD40L inhibition or knockout dramatically limits the development and progression of atherosclerosis and reduces the stability of arterial thrombi (Lutgens E. et al., Proc. Natl. Acad. Sci. USA, 2000, 97:7464-7469; Mach F et al., Nature, 1998, 394:200-203; Lutgens E. et al., Nat. Med., 1999, 5:1313-1316; Andre P. et al., Nat. Med., 1999, 5:1313-1316).

The effect of platelet inhibitors on platelet CD40L release is variable. GPIIb-IIIa antagonists inhibit CD40L release at high levels of receptor occupancy but potentiate its release at lower levels of GPIIIb-IIIa inhibition. Aspirin was shown to only partially inhibit CD40L release in collagen-stimulated platelets (Nannizzi-Alaimo L. et al., Circulation, 2003, 107:1123-1128). Platelets also synthesize and release the inflammatory cytokine IL-1β from stored messenger RNA in a GPIIb-IIIa-dependent fashion. The IL-1β is released in membrane microvesicles and induces neutrophil endothelial binding (Lindemann S. et al., J. Cell Biol, 2001, 154, 485-490). The anucleate platelet can synthesize a vast array of proteins upon activation. The β-3 subunit of GPIIb-IIIa plays an important role in the control of platelet RNA translation through the redistribution of eukaryotic initiation factor 4E, an RNA cap-binding protein that controls global translation rates (Lindermann S. et al., J. Biol. Chem. 2001, 276: 33947-33951).

### 4. Platelet Agonists

Many agonists are generated, expressed, or released at the sites of endothelial injury or activation of the coagulation cascade (see Table 1; Quinn M. et al., 2005, Platelet Function: assessment, diagnosis, and treatment, Humana Press, pp. 3-20).

TABLE 1
Platelet Agonists

Adenosine diphosphate (ADP)
Thrombin
Thromboxane A₂
Epinephrine
Serotonin
Collagen
Shear stress
Prostaglandin E₂ (PGE₂; low concentration)
8-Iso-PGF_{2α}

Platelet Agonists differ in their ability to induce platelet activation. Thrombin, collagen, and thromboxane A₂ (TXA₂) are strong agonists and can produce aggregation independent of platelet granule secretion. On the other hand, adenosine diphosphate (ADP) and serotonin are intermediate agonists and require granule secretion for full irreversible aggregation, whereas epinephrine is effective only at superphysiological concentrations.

### 4.1. Thrombin

Thrombin (factor II) is a serine protease that has diverse physiological functions. In addition to stimulating platelet activation and the conversion of fibrinogen to fibrin, it is involved in the regulation of vessel tone, smooth muscle cell proliferation and migration, inflammation, angiogenesis, and embryonic development (Wenzel U. et al., Circ Res, 1995, 77:503-509; DeMichele M. et al., J. Appl. Physiol., 1990, 69:1599-1606; Kranzholer R. et al., Circ. Res., 1996, 79:286-294; McNamara C. et al., J. Clin. Invest., 1993, 91:94-98; Haralabopoulos G. et al., Am. J. Physiol., 1997, 273:C239-C245; Griffin C. et al., Science, 2001; 293: 1666-1670; Conolly A et al., Nature, 1996, 381: 516-519). Thrombin is generated from its inactive precursor prothrombin as a result of cleavage in the coagulation cascade. This reaction is greatly facilitated by the presence of activated platelets, which supply negatively charged phospholipids for the assembly of the prothrombinase complex.

Thrombin signaling is mediated by a specialized family of G protein-linked receptors known as protease-activated receptors (PARs). Thrombin stimulates these receptors by cleaving their amino-terminal extracellular domain. This unmasks a stimulatory sequence that autostimulates the receptor (Chen J. et al., J. Biol. Chem., 1994, 269:16041-16045). Four separate PAR receptors (i.e., PARs 1-4) have been described. Three of these (PAR-1, PAR-3, and PAR-4) are activated by thrombin. PAR-2 is insensitive to thrombin but is activated by the enzyme trypsin. Two of the three PAR receptors activated by thrombin, i.e., PAR-1 and PAR-4, mediate thrombin's action on human platelets. The other one, PAR-3, is expressed only on mouse platelets (Vu T et al., Cell, 1991, 64: 1057-1068). The PAR-1 and PAR-4 receptors differ in their affinities for thrombin and the time-course of their activation and deactivation. Lower concentrations of thrombin (about 1 nM) stimulate PAR-1, and the response induced is more rapid and short-lived than the response to PAR-4 stimulation, owing to internalization of the receptor (Sharpiro M. et al., J. Biol. Chem., 2000, 275: 25216-25001). However, PAR-4 activation can also occur at low thrombin concentrations in the presence of PAR-3 due to the phenomenon of transactivation, whereby thrombin binds to PAR-3, allowing it to cleave neighboring PAR-4 (Nakanishi-Matsui M. et al., Nature, 2000, 404: 609-613). Thrombin-induced aggregation is independent on ADP secretion and Gᵢ signaling through the P2Y₁₂ ADP receptor (Kim S. et al, Blood, 2002, 99:3629-3636).

Thrombin also binds to the glycoprotein (GP)Ib-α subunit of the platelet von Willebrand factor (vWF) receptor, GPIb-IX-V, through its exocite II binding site, inducing platelet activation. This interaction, which results in allosteric inhibition of fibrinogen cleavage by thrombin, enhances thrombin's activation of PAR-1. The aggregation induced by the thrombin-GP1b interaction is dependent on platelet fibrin binding and is not inhibited by RGDS peptide (fibrinogen competing peptide, Arg-Gly-Asp-Ser) (Soslau G., et al., J. Biol. Chem., 2001, 276: 21173-21183; Li C. et al., J. Biol. Chem., 2001, 276: 6161-6168; De Candia E. et al., J. Biol. Chem., 2001, 276: 4692-4698; Quinn M. et al., 2005, Platelet Function: assessment, diagnosis, and treatment, Humana Press, pp. 3-20).

### 4.2. Collagen

Endothelial damage exposes the extracellular matrix protein collagen, which is a potent platelet agonist. Platelets have three separate surface collagen receptors, GPIa-IIa (integrin α2β1), GPVI (a member of the immunoglobulin superfamily), and GPIb-IX-V. The platelet immune receptor adaptor Fc receptor γ-chain (FcRγ) is required for collagen-induced signaling and non-covalently associates with GPVI (Tsuji M. et al., J. Biol. Chem., 1997, 272: 23528-23531). This association occurs in areas of the platelet membrane enriched with cholesterol, sphingolipids, and signaling molecules, which are known as lipid rafts (Locke D. et al., J. Biol. Chem. 2002, 277: 18801-18809). A stepwise model of activation has been proposed with initial adhesion to collagen via GPIa-IIa and subsequent interaction with GPVI/FcRγ for full platelet activation (Kebrel B. et al., Blood, 1998. 91: 491-499). Signaling involves tyrosine phosphorylation of the immunoreceptor tyrosine-based activatory motif (ITAM) of the GPIV/Fc complex by the Src family kinases Lyn and Fyn, leading to Syk binding and activation of phospholipase C. Crosslinking of the transmembrane glycoprotein platelet endothelial cell adhesion molecule-1 (PECAM-1/CD31) inhibits ITAM signaling through the PECAM-1 immunoreceptor tyrosine-based inhibitory motif (ITIM). The small guanosine triphosphatase (GTPase) RAP-1, linked to GPIIb-IIIa activation, is also activated by GPVI/Fc signaling. This is to some extent dependent on ADP secretion and signaling through P2Y₁₂. Consistent with this finding, high concentrations of collagen were shown to induce weak platelet aggregation independent of ADP release and TXA₂ production; however, maximal aggregation required TXA₂ and secretion. (Poole A. et al, EMBO J., 1997, 16: 2333-2341; Cicmil M. et al., Blood, 2002, 99: 137-144; Larson M. et al., Blood, 2003, 101: 1409-1415; Quinn M. et al., 2005, Platelet Function: assessment, diagnosis, and treatment, Humana Press, pp. 3-20).

### 4.3. Thromboxane A₂ (TXA₂)

TXA₂ is produced from arachidonic acid released from the membrane phospholipid by the action of phospholipase A₂. Arachidonic acid is metabolized to the intermediate product prostaglandin (PG)H₂ by the enzyme cyclooxygenase (COX), also referred to as PGH synthase. PGH₂ is further metabolized by a P450 enzyme, thromboxane synthase, to thromboxane A₂ or to a lesser extent in platelets, to PGE₂ (Reilly M. et al., Eur. Heart J., 1993:14:88-93; FitzGerald G., Am J. Cardiol., 1991, 68: 11B-15B). TXA₂ is labile and has a very short half-life (about 30 seconds). It is hydrolyzed rapidly to inactivate TB₂. Two separate isoforms of the TXA₂ receptor (TXRα and TXRβ) have been identified. The former is linked to Gq and the latter to Gi (Hirata T et al., J. Clin. Invest., 1996, 97, 949-956). The TXA₂ precursor, PGH₂, also stimulates these receptors (Halushka P. et al., J. Lipid Mediators Cell Signal, 1995, 12: 361-378). PGE₂ at low (nM) concentrations enhances platelet activation to subthreshold concentrations of certain agonists; conversely, at high (μM) concentrations, its actions are inhibitory (Quinn M. et al., 2005, Platelet Function: assessment, diagnosis, and treatment, Humana Press, pp. 3-20).

### 4.4. Adenosine Diphosphate

Adenosine disphosphate (ADP) is released from platelet dense granules upon activation, from red blood cells, and from damaged endothelial cells (Meyers K. et al., Am. J. Physiol., 1982, 243: R454-R461; Gardner A. et al., Nature, 1961, 192: 531-532). The platelet response to ADP is mediated by the P2Y₁ and P2Y₁₂ G protein-linked nucleotide receptors (Hollopeter G. et al., Nature, 2001, 409, 202-207; Jin J. et al., J. Biol. Chem., 1998, 273: 2030-2034). ADP also stimulates the platelet P2X₁ ligand-gated ion channel, inducing transmembrane calcium flux. P2X₁ stimulation does not play a major role in ADP-induced aggregation (Sun B. et al., J. Biol. Chem., 1998, 273, 11544-11547; Savi P. et al., Br. J. Haematol., 1997, 98, 880-886); however, low concentrations of collagen (<1 μg/mL) release ATP, which induces extracellular signal-regulated kinase (ERK)-2 activation via P2X₁ stimulation (Oury C. et al., Blood, 2002, 100:2499-2505). P2Y₁₂ stimulation is linked to inhibition of adenylate cyclase through Gi, and the Gq-coupled P2Y₁ is linked to activation of the β-isoform of phospholipase C, platelet shape change, and intracellular calcium mobilization (Hollopeter G et al., Nature, 2001, 409, 202-207; Daniel J. et al., J. Biol. Chem., 1998, 273, 2024-2029). Coordinated signaling through each receptor is required for full platelet activation and TXA₂ production (Jin J. et al., J. Biol. Chem., 1998, 273: 2030-2034; Jin J. et al., Blood, 2002, 99:193-198), although P2Y₁₂ can stimulate GPIIb-IIIa receptor activation independently at high concentrations of ADP through a phosphoinositide 3-kinase-dependent signaling pathway (Kauffenstein G. et al, FEBS Lett, 2001, 505: 281-290). The ADP receptor antagonist clopidogrel irreversibly antagonizes the P2Y₁₂ receptor, likely through the formation of a disulfide bridge between Cys 17 and Cys 270 (Ding Z. et al., Blood, 2003, 101: 3908-3914).

### 4.5. Shear Stress and Epinephrine

Epinephrine induces platelet activation through inhibition of adenylate cyclase via the Gi-linked α2-adrenergic platelet receptor. It is a weak agonist and requires other agonists to induce full platelet aggregation. High shear stress, such as that found at the site of a severe coronary stenosis, can also lead to platelet activation. High shear induces VWF to bind to GPIb-IX-V, elevating intracellular calcium and activating a protein kinase G (PKG) signaling pathway that leads to mitogen-activated protein (MAP) kinase and GPIIb-IIIa activation (Li Z. et al., J. Biol. Chem., 2001, 276: 42226-42232).

Among the various platelet stimulants, thrombin is the most potent activator of platelets. Vascular injury and inflammation expose tissue factor, resulting in the formation of a tissue/factor/factor VIIa complex that leads to the local generation of thrombin from prothrombin. Platelets also facilitate thrombin generation by providing procoagulant phospholipid surfaces that anchor various coagulation factors. Thrombin activates platelets at very low concentrations by interacting with PARs (Barrish, J. et al., Accounts in Drug Discovery: Case Studies in Medicinal Chemistry, 2010, Royal Society of Chemistry, 2010).

Activation of platelets and the ensuing intracellular biochemical events lead to the activation of surface GP IIb/IIIa receptors, which is the final, convergent pathway in the platelet activation mechanism. GP IIb/IIIa is a member of the integrin family of receptors, composed of α and symbols (α_{IIb}β₃). In the resting state, platelet GP IIb/IIIa receptors do not bind to fibrinogen (or bind with a very low affinity) (Hirata T et al., J. Clin. Invest., 1996, 97: 949-956). Activation alters the conformation of GP II/IIIa, rendering it capable of binding to extracellular macromolecular ligands, including fibrinogen and von Willebrand Factor (vWF). The arginine-glycine-aspartic acid (RGD) sequence of the adhesive proteins binds to the GP IIb/IIIa receptor. Fibrinogen contains two RGD sequence on its a chain, one in the N-terminal region and the other in the C-terminal region, and is therefore bivalent in its binding to GP IIb/IIIa receptors, which allows efficient cross-linking of platelets. Although vWF also binds to the GP IIb/IIIa receptor at its various RGD sites, studies in fibrinogen knockout mice have shown that vWF alone is not sufficient to achieve stable platelet aggregation (Ni, H. et al., J. Clin. Invest., 2000, 106, 385; Barrish, J et al., Accounts in Drug Discovery: Case Studies in Medicinal Chemistry, 2010, Royal Society of Chemistry, 2010).

### 5. Platelet Secretion

Platelets release a number of biologically active substances upon activation (see Table 2; Quinn M. et al., 2005, Platelet Function: assessment, diagnosis, and treatment, Humana Press, pp. 3-20).

TABLE 2
Platelet Secretions.
Platelet Secretions

Platelet-derived growth factor
P-selectin
RANTES
Platelet-activating factor
β-Thromboglobulin
Platelet factor 4
von Willebrand factor
α₂-Antiplasmin
Coagulation factor V
Fibrinogen
Fibronectin
Thrombospondin
ADP
Serotonin
CD40L
Matrix metalloproteinases 1 and 2
Vascular endothelial growth factor
Insulin-like growth factor
Epidermal and fibroblast growth factors
Transforming growth factor-β₁

These include the contents of their α and dense granules, lysozymes, and platelet-derived microparticles. In addition, activated platelets synthesize and secrete a number of biologically active products and express the inflammatory stimulant CD40L (Henn V. et al., Nature, 1998, 391:591-594). Platelet α-granules contain platelet-derived growth factor, P-selectin, vWF, α2-antiplasmin, β-thromboglobulin, platelet factor 4 coagulation factor V, and adhesion molecules, such as fibrinogen, fibronectin, and thrombospondin; the dense granules contain ADP and serotonin. The released ADP provides a feedback loop for further platelet stimulation, and serotonin enables the binding of some of the proteins released from the α-granules to a subpopulation of platelets, through an as yet undefined receptor (Dale, G. et al., Nature, 2002, 415: 175-179). Platelet secretion requires the formation of soluble N-ethylmaleimide-sensitive factor attachment protein (SNAP) and receptor (SNARE) complexes among syntaxin 4, SNAP-25, and vesicle-associated membrane proteins (VAMP-3 and VAMP-8) (Polgar J. et al, Blood, 2002, 100: 1081-1083; and Quinn M. et al., 2005, Platelet Function: assessment, diagnosis, and treatment, Humana Press, pp. 3-20).

Activated platelets also release membrane microparticles. These contain GPIIb-IIIa, thrombospondin, and P-selectin, enhance local thrombin generation, and induce COX-2 expression with the production of prostacyclin in monocytes and endothelial cells.

### 6. Chemokine

Chemokines (chemotactic cytokines) are a small group of proteins with four conserved cysteines forming two essential disulfide bonds. CXC and CC chemokines are distinguished according to the position of the first two cysteines, which are either adjacent (CC or β chemokines) or separated by one amino acid (CXC or α chemokines). Recently, lymphotactin, a chemokine with only two conserved cysteines(C), as well as chemokines with three amino acids between the first two cysteines (CX₃C motif) have been described. Chemokines can be divided further in a number of ways based on differing functional parameters. These include their ability to initiate inflammatory versus homeostatic migration and/or recirculation of lymphocytes, granulocytes, and mononuclear cells (homeostatic vs. inflammatory/inducible). Other delineations include, for instance, an ability to promote or inhibit angiogenesis (ELR vs non ELR CXC chemokines) (Harrison J. and Lukacs N., 2007, The Chemokine receptors, Humana Press, pp. 1-8).

In general, CC chemokines chemoattract monocytes, basophils, eosinophils, and T-lymphocyte, but not neutrophils. Members of this family include monocyte chemoattractant protein-1 (MCP-1/CCL2), macrophage inhibitory protein-1α (MIP-1α/CCL3), macrophage inhibitory protein-1β (MIP-1β/CCL4), and the chemokine, Regulated upon Activation, Normal T-cell Expressed and Secreted (RANTES/CCL5). Lymphotactin/XCL1 (a chemoattractant for T-lymphocytes) and fractalkine/CX3CL1 (a chemoattractant for T-lymphocytes and monocytes) are members of the C and CX₃C subfamilies, respectively.

IL-8 belongs to the C—X—C subfamily of chemokines, which displays four highly conserved cysteine amino acid residues, with the first two separated by one nonconserved residue. In contrast, MCP-1 and RANTES belong to the C—C subfamily, which exhibits two adjacent cysteine amino acid residues. The chemokine subfamilies exhibit cell selectivity with respect to chemoattraction. Members of the C—X—C subfamily primarily target neutrophils, whereas various members of the C—C subfamily target monocytes, lymphocytes, eosinophils, and basophils. Specifically, IL-8 mediates neutrophil chemotaxis, whereas MCP-1 mediates monocyte and basophil chemotaxis and activation.

Although the regulation of leukocyte migration and activation during immune responses is the major function of chemokines, they are also thought to modulate additional biological activities, including hematopoiesis, apoptosis, angiogenesis, cell proliferation, and viral pathogenesis. A listing of some chemokines and chemokine receptors that have been identified is shown in Table 3 (Schwiebert, L., 2005, Chemokines, Chemokine Receptors, and Disease, Chemokine and Receptor families, Gulf Professional Publishing, pp. 1-46).

TABLE 3
Chemokine and Receptor Families

| Systematic | | | |
|---|---|---|---|
| nomenclature | Common nomenclature | Chemokine receptor(s) | |
| CXC Family | | | |
| CXCL1 | Groz/MGSA-z | CXCR2 | □ > CXCR |

| | | |
|---|---|---|
| CXCL2 | Groβ/MGSA-β | CXCR2 |
| CXCL3 | Groγ/MGSA-γ | CXCR2 |
| CXCL4 | PF4 | Unknown |
| CXCL5 | ENA-78 | CXCR2 |
| CXCL6 | GCP-2 | CXCR1, CXCR2 |
| CXCL7 | NAP-2 | CXCR2 |
| CXCL8 | IL-8 | CXCR1, CXCR2 |
| CXCL9 | Mig | CXCR3 |
| CXCL10 | IP-10 | CXCR3 |
| CXCL11 | I-TAC | CXCR3 |
| CXCL12 | SDF-lx/β | CXCR4 |
| CXCL13 | BLC/BCA-1 | CXCR5 |
| CXCL14 | BRAK | Unknown |
| CXCL15 | Lungkine | Unknown |
| CXCL16 | SR-PSOX | CXCR6 |
| CC Family | | |
| CCL1 | I-309 | CCR8 |
| CCL2 | MCP-1/MCAF/TDCF | CCR2 |
| CCL3 | MIP-1x/LD78x | CCR1, CCR5 |
| CCL4 | MIP-1β | CCR5 |
| CCL5 | RANTES | CCR1, CCR3, CCR5 |
| CCL6 | C10, MRP-1 | CCR1? |
| CCL7 | MCP-3 | CCR1, CCR2, CCR3 |
| CCL8 | MCP-2 | CCR1, CCR2, CCR3, CCR5 |
| CCL9/10 | MRP-2, CCF18, MIP-1γ | CCR1 |
| CCL11 | Eotaxin | CCR3 > CCR5 |
| CCL12 | MCP-5 | CCR2 |
| CCL13 | MCP-4 | CCR2, CCR3 |
| CCL14 | HCC-1 | CCR1, CCR3, CCR5 |
| CCL15 | HCC-2/Lkn-1/MIP-1 [symbol] | CCR1, CCR3 |
| CCL16 | HCC-4/LEC/LCC-1 | CCR1, CCR2, CCR5, CCR8 |
| CCL17 | TARC | CCR4 |
| CCL18 | DC-CK1/PARC/ | Unknown |
| | AMAC-1 | |

Most chemokines are produced under pathological conditions by tissue cells and infiltrating leukocytes. Stimulation of leukocyte suspensions with chemokines leads to a fast shape change that involves polymerization of actin filaments, formation of lamellipodia and activation of the integrins that mediate adhesion to endothelial cells. Moreover, the activation of leukocytes by cytokines induces other responses, such as, without limitation, the rise in intracellular calcium concentration, the production of oxygen radicals and bioactive lipids, and the release of the content of cytoplasmic storage granules, such as proteases from neutrophils and monocytes, histamine from basophils, or cytotoxic protein from eosinophils. The effects of chemokines are mediated by seven transmembrane domain receptors coupled to GTP-binding proteins. The main sites of interaction for chemokines are with their receptor in the N-terminal region and within an exposed loop of the backbone that extends between the second and the third cysteine. The N-terminal binding site is important for the triggering of the receptor.

### 7. General Features of Chemokine Receptors

Known chemokine receptors can be grouped into four different classes: the “specific”, “shared”, “promiscuous”, and “virally encoded” receptors.

“Specific” chemokine receptors bind only one chemokine. To date, a specific RANTES chemokine receptor has not been described.

“Shared” chemokine receptors bind more than one chemokine within either the C—X—C or the C—C subfamily. The chemokine receptors CCR1, CCR3, and CCR5 all bind RANTES, and other C—C chemokines, and thus fall into the shared category.

The Duffy antigen receptor for chemokines is a promiscuous chemokine receptor that binds both C—X—C and C—C chemokines including RANTES.

The fourth type of chemokine receptor is encoded within viral genomes.

Chemokine receptors have multiple functions. Not only do they mediate chemotaxis but they also mediate the upregulation of integrins, actin polymerization, respiratory burst, degranulation, and cell proliferation. Most of the biological effects of chemokines are mediated primarily through interactions with different classes of G-protein-coupled receptors that span the membrane seven times (also called as serpentine receptors). The chemokine receptor genes are expressed in a cell type-specific manner and this differential expression is the basis for the specificity of chemokines for subsets of leukocytes.

The second and third intracellular loops of serpentine receptors interact with a G-protein heterotrimer and, upon ligand binding, exchange GDP for GTP, resulting in activation of the G-protein subunits (Neote and McColl, C—C chemokine receptors. In “Chemoattractant Ligands and Their Receptors, CRC press, 1996). In turn, the activated G-proteins signal effector enzymes, such as phospholipase Cβ2. GTP is hydrolyzed to GDP, and the GDP from of the G protein completes the cycle by complexing with unoccupied receptors. Most known biological effects of chemokines are inhibited by pertussis toxin. Pertussis toxin causes ADP-ribosylation of the Gal subunits and thus irreversibly inactivates their action

Serine and threonine amino acid residues found at the carboxyl terminus of the serpentine receptors act as substrates for phosphorylation. Phosphorylation of the carboxyl terminus of the serpentine receptors leads to binding of arrestin, which prevents binding of G-proteins to the receptor and hence prevents signaling. This may be the mechanism of desensitization by which prior exposure to a ligand blocks the subsequent response to the same ligand. Desensitization causes the rapid cessation of the ligand-induced responses critical to the cellular response to a concentration gradient, and thus inhibits chemotaxis (Murphy et al., Annu Rev. Immunol., 1994, 12, 593-633).

### 8. RANTES (Regulated on Activation, Normal T-Cell Expressed and Secreted)

RANTES (also known as CCL5) was identified originally as a DNA during a general screen for genes selectively expressed by functionally mature cytotoxic T lymphocytes and not by B cells. The name RANTES is an acronym derived from the original observed and predicted characteristics of the gene and the protein it encodes: Regulated upon Activation Normal T cell Expressed and Secreted.

Subsequently, it was shown that the RANTES protein could function as a potent chemoattractant for monocytes, specific subsets of T lymphocytes, eosinophils, basophils, and natural killer cells. RANTES can also cause degranulation of basophils, respiratory burst in eosinophils, and the stimulation of T cell proliferation. RANTES may also have antiviral properties, as it has been shown to suppress replication of HIV in vitro (Knobli, K. et al., 1998, Am. J. Physiol. 272: L134). In vivo, RANTES is expressed in diseases characterized by a mononuclear cell infiltration, such as renal allograft rejection, delayed type hypersensitivity, and inflammatory lung disease. These multiple activities suggest a role for the RANTES chemokine in both acute and chronic phases of inflammation (Mire-Sluis A. and Thorpe R., 1998, Cytokines, Academic Press, pp. 432-445).

Expression of RANTES can be induced in a wide variety of cell types including T cells, monocytes, basophils, mesangial cells, fibroblasts, epithelial cells, and endothelial cells. Megakaryocytes, which form platelets, make RANTES constitutively. The rapid expression of RANTES by fibroblasts, endothelial cells, and epithelial cells may be an early response to stress by injured tissue. It has been speculated that this expression results in the attraction and infiltration of a variety of inflammatory cell types, including monocytes and memory T cells, into the stressed tissue. It was suggested that the expression of RANTES, accompanying T cell and monocyte effector function, may represent means to amplify and propagate an inflammatory response (Mire-Sluis A. and Thorpe R., 1998, Cytokines, Academic Press, pp. 432-445).

### 9. RANTES Receptors

To date, a specific RANTES chemokine receptor has not been described. The chemokine receptors CCR1, CCR3, and CCR5 all bind RANTES and other C—C chemokines, and thus fall into the shared category. The US28 gene found in the cytomegalovirus (CMV) genome binds the RANTES protein. In addition to specific high-affinity receptors on leukocytes and erythrocytes, RANTES also binds to proteoglycans on the endothelium that can present the chemokine to circulating leukocytes (Mire-Sluis A. and Thorpe R., 1998, Cytokines, Academic Press, pp. 432-445).

In vivo, RANTES protein has been localized to the endothelium and extracellular matrix of the microvasculature during inflammation. In this regard, it is ideally located to function as a haptotactic agent (Mire-Sluis A. and Thorpe R., 1998, Cytokines, Academic Press, pp. 432-445). It was reported that RANTES is a potent haptotactic and chemotactic agent for monocytes. Chemotaxis along soluble gradients in blood vessels is unlikely since a soluble gradient would be quickly dispersed by the blood. Therefore, haptotaxis, which is defined as cell migration induced by surface bound gradients of chemoattractants, along a gradient of agent bound to the endothelial surface can be more effective in vivo.

### 10. Molecular Mechanisms of RANTES Regulation

With regard to signaling mechanisms that regulate inflammatory cytokines, a recent study has suggested that p38 MAP kinase and the c-Jun-NH₂ terminal kinase (JNK)-dependent pathway are involved in RANTES production by influenza virus-infected human bronchial epithelial cells (Kujime et al., J. Immunol., 2000, 164: 3222-3228).

A mammalian MAP kinase superfamily has been molecularly characterized: extracellular signal-regulated kinase (Erk), p38 MAP kinase, and c-Jun-NH₂-terminal kinase (JNK). p38 MAP kinase and JNK are activated by environmental stresses such as hyperosmotic shock, heat shock, cold shock, UV irradiation, and inflammatory cytokines and play an important role in apoptosis and cytokine expression, whereas Erk is activated by mitogen stimuli and plays a central role in cell proliferation and differentiation; however, recent studies have suggested that Erk and JNK also play an important role in the signal cascades of induction of various inflammatory mediators including cytokines and chemical mediators (Trotta, R. et al., J. Exp. Med., 1996, 184:1027; Rose, D. et al., J. Immunol., 1997 158: 3433; Zhang, C. et al., J. Biol. Chem., 1997, 272:13397; Bhat, N. et al, J. Neurosci., 1998, 18:1633; Rawadi, G., J. Immunol., 1998, 160:1330; and Tuyt, L. et al., J. Immunol., 1999, 162:4893).

MAP kinase cascades are connected with the activation of various transcription factors that participate to various extents in the inducible expression of gene-encoding cytokine. The promoter of the gene-encoding RANTES contains sequences for the binding several nuclear transcription factors including NF-kB and AP-1 (Bauerle, P. and Henkel, T., Annu Rev. Immunol., 1994, 12:141; Nelson, P. et al., J. Immunol., 1993, 151:2601). These transcription factors participate to various extents in the inducible expression of the gene encoding RANTES. p38 MAP kinase has been implicated in the activation of multiple transcription factors, including NF-kB (Wesseborg, S. et al., J. Biol. Chem., 1997, 272:12422). JNK has been implicated in the activation of multiple transcription factors, including AP-1. For example, it has been shown that JNK and NF-kB response elements are involved in RANTES gene activation in a macrophage cell line, RAW 264.7 cells, stimulated by LPS (Hiura, T. et al., Clin. Immunol., 1999, 90:287).

It has been reported that expression of inflammatory cytokines activated by MAP kinase signaling cascade can be inhibited by an antioxidative agent, such as N-acetylcysteine (NAC). For example, it was shown that N-acetylcysteine can inhibit interluekin-1 beta (IL-1β)-induced eotaxin and monocyte chemotactic protein-1 (MCP-1) expression and production by inhibiting activation of p38 MAP kinase, opening up a possibility that an elevation of a RANTES level in other tissues may also be regulated by NAC (Wuyts, W. et al., Eur. Respr. J., 2003, 22: 43-49).

Other studies have shown that diesel exhaust particles induce IL-8 and RANTES production and the threonine and tyrosine phosphorylation of p38 MAP kinase, reflecting the activation of p38 MAP kinase in human bronchial epithelial cells. N-acetylcysteine was shown to inhibit diesel exhaust particle (DEP)-induced p38 MAP kinase activity, and inhibited DEP-induced IL-8 and RANTES production. These results suggest that the p38 MAP kinase signaling pathway plays an important role in the DEP-activated signaling pathway that regulates IL-8 and RANTES production by bronchial epithelial cells and that the cellular redox state is critical for DEP-induced p38 MAP kinase activation leading to IL-8 and RANTES production.

### 11. Fibrosis

Fibrosis is the formation or development of excess fibrous connective tissue in an organ or tissue as a result of injury, inflammation, or of interference with the blood supply. It may be a consequence of the normal wound healing response leading to a scar, or it may be an abnormal, reactive process. There are several types of fibrosis including, but not limited to, cystic fibrosis of the pancreas and lungs, injection fibrosis, endomyocardial fibrosis, mediastinal fibrosis, myelofibrosis, retroperitoneal fibrosis, nephrogenic systemic fibrosis, and idiopathic pulmonary fibrosis of the lung.

### 12. Cystic Fibrosis

Cystic fibrosis (CF, mucovisidosis) is an inherited autosomal recessive disorder. It is one of the most common fatal genetic disorders in the United States, affecting about 30,000 individuals, and is most prevalent in the Caucasian population, occurring in one of every 3,300 live births. The gene involved in cystic fibrosis, which was identified in 1989, codes for a protein called the cystic fibrosis transmembrane conductance regulator (CFTR). CFTR normally is expressed by exocrine epithelia throughout the body and regulates the movement of chloride ions, bicarbonate ions and glutathione into and out of cells. In cystic fibrosis patients, mutations in the CFTR gene lead to alterations or total loss of CFTR protein function, resulting in defects in osmolarity, pH and redox properties of exocrine secretions. In the lungs, CF manifests itself by the presence of a thick mucus secretion which clogs the airways. In other exocrine organs, such as the sweat glands, CF may not manifest itself by an obstructive phenotype, but rather by abnormal salt composition of the secretions (hence the clinical sweat osmolarity test used to detect CF patients).

The predominant cause of illness and death in cystic fibrosis patients is progressive lung disease. The thickness of CF mucus, which blocks the airway passages, is believed to stem from abnormalities in osmolarity of secretions, as well as from the presence of massive amounts of DNA, actin, proteases and prooxidative enzymes originating from a subset of inflammatory cells, called neutrophils. Indeed, CF lung disease is characterized by early, hyperactive neutrophil-mediated inflammatory reactions to both viral and bacterial pathogens.

The hyperinflammatory syndrome of CF lungs has several underpinnings, among which an imbalance between pro-inflammatory chemokines, chiefly IL-8, and anti-inflammatory cytokines, chiefly IL-10, has been reported to play a major role (Chmiel et al., 2002, Clin Rev Allergy Immunol. 3(1):5-27). Studies have reported that levels of TNF-α, IL-6 and IL-1β were higher in the bronchoalveolar lavage fluid of cystic fibrosis patients than in healthy control bronchoalveolar lavage fluid (Bondfield, T. et al., 1995, Am. J. Resp. Crit. Care Med. 152(1): 2111-2118).

The hyperinflammatory syndrome at play in CF lungs may predispose such patients to chronic infections with colonizing bacterial pathogens. The most common bacterium to infect the CF lung is Pseudomonas aeruginosa, a gram-negative microorganism. The lungs of most children with CF become colonized by P. aeruginosa before their third birthday. By their tenth birthday, P. aeruginosa becomes dominant over other opportunistic pathogens (Gibson et al., 2003, Am. J. Respir. Crit. Care Med., 168(8): 918-951). P. aeruginosa infections further exacerbate neutrophilic inflammation, which causes repeated episodes of intense breathing problems in CF patients. Although antibiotics can decrease the frequency and duration of these attacks, the bacterium progressively establishes a permanent residence in CF lungs by switching to a so-called “mucoid,” biofilm form of high resistance and low virulence, which never can be eliminated completely from the lungs. The continuous presence in CF lungs of inflammatory by-products, such as extracellular DNA and elastase, could play a major role in selecting for mucoid P. aeruginosa forms (Walker et al., 2005, Infect Immun. 73(6): 3693-3701).

Treatments for CF lung disease typically involve antibiotics, anti-inflammatory drugs, bronchodilators, and chest physiotherapy to help fight infection, neutrophilic inflammation and obstruction and clear the airways. Nevertheless, the persistent, viscous and toxic nature of airway secretions in cystic fibrosis lung disease still leads to progressive deterioration of lung function (Rancourt et al., 2004, Am. J. Physiol. Lung Cell Mol. Physiol. 286(5): L931-38).

### 13. RANTES and Cystic Fibrosis

The cystic fibrosis (CF) lung disease phenotype includes thick mucus secretion and bacterial colonization of the airway with Pseudomonas aeruginosa. Recent reports suggest that the CF lung may exhibit an exaggerated immune response, even in the absence of bacterial infection. CF-associated airway inflammation is characterized by a profound influx of neutrophils into the lung; however, other types of leukocytes, including eosinophils and monocytes, have been implicated in CF airway inflammation. (Schwiebert, L. et al., Am J. Physiol. Cell Physiol. 276: C700-C710).

Airway epithelial cells have been described classically as barrier cells that are involved in ion and fluid homeostasis. These cells respond to a variety of environmental stimuli, resulting in the alteration of their cellular functions, such as ion transport and movement of airway secretions. In addition, airway epithelial cells may act as immune effector cells in response to endogenous or exogenous stimuli. Several studies have shown that airway epithelial cells express and secrete a variety of inflammatory mediators, including the chemokines, such as interleukin-8 (IL-8), monocyte chemoattractant protein-1 (MCP-1), and RANTES.

RANTES induces chemotaxis of eosinophils, monocytes, and CD45 RO+ memory T lymphocytes. The expression of chemokines by airway epithelial cells implicates these cells in facilitating the leukocyte migration associated with airway inflammatory diseases such as CF (Schwiebert, L. et al., Am J. Physiol. Cell Physiol. 276:C700-C710).

### 14. Autism

Autism is the most severe and devastating condition in the broad spectrum of developmental disorders called “pervasive developmental disorders” (Rapin, I., 1997, New Engl. J. Med., 337: 97-104). Autistic disorders are characterized by marked impairment in social skills, verbal communication, behavior, and cognitive function (Rapin, I. 1997, New Engl. J. Med., 337: 97-104; Lord, C. et al., 2000, Neuron, 28, 355-363). Abnormalities in language development, mental retardation, and epilepsy are frequent problems in the clinical profile of patients with autism. The syndrome is clinically heterogeneous and can be associated in up to 10% of patients with well-described neurological and genetic disorders, such as tuberous sclerosis, fragile X, Rett and Down syndromes, although in most patients the causes are still unknown (Rapin, I., and Katzman R., 1998, Annals of Neurology, 43, 7-14; Newschaffer, C. et al., 2002, Epidemiology Reviews, 24, 137-153; Cohen, D. et al., 2005, Journal of Autism & Developmental Disorders, 35, 103-116).

### 14.1. Neurobiology of Autism

### 14.2. Clinical and Epidemiological Aspects of Autism

Although the neurobiological basis for autism remains poorly understood, several lines of research now support the view that genetic, environmental, neurological, and immunological factors contribute to its development (Rapin, I., and Katzman R., 1998, Annals of Neurology, 43, 7-14; Newschaffer, C. et al., 2002, Epidemiology Reviews, 24, 137-153; Folstein, S. and Rosen-Sheidley, B., 2001, Nature Reviews Genetics, 2, 943-955; Korvatska, E., et al., 2002, Neurobiology of Disease, 9, 107-125). Several different genetic factors and/or other risk factors may combine during development to produce complex changes in CNS organization that translate into abnormalities of neuronal and cortical cytoarchitecture that are responsible for the complex language and behavioral problems that characterize the autistic phenotype. The core symptoms of autism include abnormal communication, social relatedness, behavior, and cognition (Rapin, I., 1997, N. Engl. J. Med., 337: 97-104; Lord, C. et al., 2000, Neuron, 28, 355-363).

The majority of autistic children show abnormalities during infant development that may not become apparent until the second year of life. Approximately 30-50% of children undergo regression, with a loss of skills, including language, between 16 and 25 months of age. In the medical evaluation of autism, specific etiologies can be found in <10% of children, including fragile X, tuberous sclerosis, and other rare diseases (Cohen, D. et al., 2005, Journal of Autism & Developmental Disorders, 35, 103-116). Epilepsy occurs in up to 40% of patients, and epileptic discharges may occur on EEGs early in childhood, even in the absence of clinical seizures (Tuchman, R. and Rapin, I., 2002, Lancet Neurology, 1, 352-358). Although children with autism present with a wide spectrum of symptoms that vary in severity and clinical progression, it is possible to define these features in affected individuals and follow them over time (Aman, M. et al., 2004, CNS Spectrums, 9, 36-47).

### 14.3. Neuroanatomical Abnormalities in Autism

A wide range of anatomical and structural brain abnormalities have been observed in autistic patients by longitudinal clinical and magnetic resonance imaging studies. The clinical onset of autism appears to be preceded by two phases of brain growth abnormalities: a reduced head size at birth and a sudden and excessive increase in head size between 1-2 months and 6-14 months (Courchesne et al., 2004, Curr. Opin. Neurol., 17, 489-496). These studies have also shown that the most abnormal pattern of brain overgrowth occurs in areas of the frontal lobe, cerebellum, and limbic structures between 2-4 years of age, a pattern that is followed by abnormal slowness and an arrest in brain growth (Courchesne et al., 2004, Curr. Opin. Neurol., 17, 489-496; Courchesne, E. and Pierce, K., 2005, International Journal of Developmental Neuroscience, 23, 153-170). Other studies of high-functioning autistic patients have shown an overall enlargement of brain volume associated with increased cerebral white matter and decrease in cerebral cortex and hippocampal-amygdala volumes (Herbert et al., 2003, Brain, 126, 1182-1192.; Herbert et al., 2004, Annals of Neurology, 55, 530-540). However, the cause of this dissociation or patterns of abnormal brain growth is not understand completely.

Other studies have shown that disruption of white matter tracts and disconnection between brain regions are present in autistic patients, as demonstrated by new techniques such as diffusion tensor imaging. This approach has demonstrated reduced fractional anisotropy values in white matter adjacent to the ventromedial prefrontal cortices, anterior cingulate gyrus, and superior temporal regions, findings suggestive of the disruption in white matter tracts in brain regions involved in social functioning that has been described in autistic patients (Barnea-Goraly et al., 2004, Biological Psychiatry, 55, 323-326).

In addition to abnormal growth patterns of the brain, one of the most consistent findings of neuroimaging studies in autism is the presence of abnormalities in the cerebellum. Reduction in the size of cerebellar regions such as the vermis (Hashimoto et al., 1995, Journal of Autism & Developmental Disorders, 25, 1-18; Kaufmann et al., 2003, Journal of Child Neurology, 18, 463-470), an increase in white matter volume, and reduction in the gray/white matter ratio (Courchesne, E. and Pierce, K., 2005, International Journal of Developmental Neuroscience, 23, 153-170) are the most prominent changes observed in the cerebellum. In one of these studies, the cerebellar changes appeared to be specific to autism, in contrast to other neurodevelopmental disorders such as Down syndrome, Down syndrome with autism, fragile X and fragile X with autism (Kaufmann et al., 2003, Journal of Child Neurology, 18, 463-470). These observations concur with: (1) the findings from neuropathological studies describing abnormalities in the cerebellum, such as a decreased number of Purkinje cells (Kemper, T. and Bauman, M., 1998, Journal of Neuropathology & Experimental Neurology, 57, 645-652; Bailey et al., 1998, Brain, 121(Pt 5), 889-905.) and, most recently, (2) observation of increased microglial activation and astroglial reactions in both the granular cell and white matter layers and a reduction in Purkinje and granular cells (Vargas et al., 2005, Annals of Neurology, 57, 67-81).

### 14.4. Neuropathology of Autism

Cytoarchitectural organizational abnormalities of the cerebral cortex, cerebellum, and other subcortical structures are the most prominent neuropathological changes in autism (Kemper, T. and Bauman, M., 1998, Journal of Neuropathology & Experimental Neurology, 57, 645-652; Bailey et al., 1998, Archives of Pediatric & Adolescent Medicine, 159, 37-44). An unusual laminar cytoarchitecture with packed small neurons has been described in classical neuropathological studies, but no abnormalities in the external configuration of the cerebral cortex were noted (Kemper, T. and Bauman, M., 1998, Journal of Neuropathology & Experimental Neurology, 57, 645-652). Cerebellar and brainstem pathology was prominent, with a loss and atrophy of Purkinje cells, predominantly in the posterior lateral neocerebellar cortex. At least three different types of pathological abnormalities have been delineated in autism: (1) a curtailment of the normal development of neurons in the forebrain limbic system; (2) an apparent decrease in the cerebellar Purkinje cell population; and (3) age related changes in neuronal size and number in the nucleus of the diagonal band of Broca, the cerebellar nuclei, and the inferior olive (Kemper, T. and Bauman, M., 1998, Journal of Neuropathology & Experimental Neurology, 57, 645-652). These observations suggest that delays in neuronal maturation are an important component in the spectrum. In addition to these cytoarchitectural abnormalities, the number of cortical minicolumns, the narrow chain of neurons that extend vertically across layers 2-6 to form anatomical and functional units, appeared to be more numerous, smaller, and less compact in their cellular configuration in the frontal and temporal regions of the brain of autistic patients, as compared with controls (Casanova et al., 2002, Neurology, 58, 428-432). Pathological evidence of immunological reactions within the CNS, such as lymphocyte infiltration and microglial nodules, has been described in a few case reports (Bailey et al., 1998, Brain, 121(Pt 5), 889-905; Guerin et al., 1996, Developmental Medicine & Child Neurology, 38, 203-211).

### 14.5. Immunological Abnormalities in Autism

Reports of differences in systemic immune findings over the past 30 years have led to speculation that autism may represent, in some patients, an immune mediated or autoimmune disorder (Ashwood, P. and Van de Water, J., 2004, Autoimmunity Reviews, 3, 557-562). Recent studies of immune dysfunction in autism have sought to understand these findings in the clinical context of the syndrome (Korvatska et al., 2002, Neurobiology of Disease, 9, 107-125; Ashwood, P. and Van de Water, J., 2004, Autoimmunity Reviews, 3, 557-562; Zimmerman, 2005, The immune system. In M. Bauman & T. L. Kemper (Eds.), The Neurobiology of Autism pp. 371-386, The Johns Hopkins University Press). Abnormalities of both humoral and cellular immune functions have been described in small studies of children with autism (N=10-36), and include decreased production of immunoglobulins or B and T-cell dysfunction (Warren et al., 1986, Journal of Autism & Developmental Disorders, 16, 189-197). Early studies suggested that prenatal viral infections might damage the immature immune system and induce viral tolerance (Stubbs, E. and Crawford, M., 1977, Journal of Autism & Child Schizophrenia, 7, 49-55), while later studies showed altered T-cell subsets and activation, consistent with the possibility of an autoimmune pathogenesis (Gupta et al., 1998, Journal of Neuroimmunology, 85, 106-109). Recently, earlier reports of a four-fold increase in the serum complement (C4B) null allele (i.e., no protein produced) was confirmed in 85 children with autism, compared to controls.

Studies of peripheral blood have shown a range of abnormalities, including T-cell, B-cell, and NK-cell dysfunction; autoantibody production; and increased pro-inflammatory cytokines (Gupta et al., 1998, Journal of Neuroimmunology, 85, 106-109; Singh et al., 1997, Pediatric Neurology, 17, 88-90; Singh et al., 2002, Journal of Biomedical Science, 9, 359-364; Vojdani et al., 2002, Journal of Neuroimmunology, 129, 168-177; Jyonouchi et al., 2001, Journal of Neuroimmunology, 120, 170-179). Shifts observed in Th1 to Th2 lymphocyte subsets and cytokines and associations with human leukocyte antigen (HLA)-DR4 have suggested the possibility that autoimmunity against brain antigens may contribute to the neuropathology of autism (van Gent et al., 1997, Journal of Child Psychology & Psychiatry, 38, 337-349).

Decreases in immunoglobulin subsets and complement, the presence of auto-antibodies against CNS antigens, and an effect of maternal antibodies have also been proposed as pathogenic factors (Dalton et al., 2003, Annal of Neurology, 53, 533-537). In most of these studies, phenotyping was limited to descriptions of the subjects as “autistic” based on criteria of the Diagnostic and Statistical Manual of the American Psychiatric Association. “Abnormal” immune findings varied from 15-60% of children with autism. For some parameters, unaffected siblings showed intermediate values, and a background of such “abnormalities” was noted in normal controls as well. In all studies, measurements have been reported at single time points and among subjects of different ages. Since these differences in systemic immune findings in autism have not been followed in the same patients over time, it is not clear whether they reflect true immune dysfunction or represent dysmaturation that changes with age (Zimmermann, 2005, The Neurobiology of Autism, pp. 371-386, The Johns Hopkins University Press). Also, no clinical immunodeficiency states have been reported in association with unusual infections or reactions to immunizations, despite widespread interest in the possibility of such relationships (Halsey, N. and Hyman, S., 2001, Pediatrics, 107, E84).

### 14. 6. Immune-to-Brain Communication Pathways

The brain has long been considered an ‘immune-privileged’ organ but this immune status is far from absolute and varies with age and brain region. Moreover, the brain contains immune cells, such as macrophages and dendritic cells, which are present in the choroid plexus and meninges. Brain parenchymal macrophages, known as microglial cells, are more quiescent in comparison with other tissue macrophages but can respond to inflammatory stimuli by producing pro-inflammatory cytokines and prostaglandins. In addition, both neuronal and non-neuronal brain cells express receptors for these mediators (Dantzer et al., Nat Rev Neurosci., 2008, 9: 46-56).

The brain monitors peripheral innate immune responses by several means that act in parallel (FIG. 3). One pathway involves afferent nerves: locally produced cytokines activate primary afferent nerves, such as the vagal nerves during abdominal and visceral infections and the trigeminal nerves during oro-lingual infections. In a second, humoral pathway, Toll-like receptors (TLRs) on macrophage-like cells residing in the circumventricular organs and the choroid plexus respond to circulating pathogen-associated molecular patterns by producing pro-inflammatory cytokines. As the circumventricular organs lie outside the blood-brain barrier, these cytokines can enter the brain by volume diffusion. A third pathway comprises cytokine transporters at the blood-brain barrier: pro-inflammatory cytokines overflowing in the systemic circulation can gain access to the brain through these saturable transport systems. Finally, a fourth pathway involves IL-1 receptors that are located on perivascular macrophages and endothelial cells of brain venules. Activation of these IL-1 receptors by circulating cytokines results in the local production of prostaglandin E2.

Engagement of these immune-to-brain communication pathways ultimately leads to the production of pro-inflammatory cytokines by microglial cells. This process requires the convergent action of two events with different time courses: the activation of the rapid afferent neural pathway, and a slower propagation of the cytokine message within the brain. Activation of the neural pathway (FIG. 3) probably sensitizes target brain structures for the production and action of cytokines that propagate from the circumventricular organs and the choroid plexus into the brain. This way the brain forms an ‘image’ of the peripheral innate immune response that is similar in its elementary molecular components to the response in the periphery. The main difference is that this brain image does not involve an invasion of immune cells into the parenchyma and is not distorted by tissue damage that occurs at the site of infection.

The brain circuitry that mediates the various behavioral actions of cytokines remains elusive. The social withdrawal that characterizes cytokine-induced sickness behavior is unlikely to be mediated by the same brain areas as those underlying other responses to infection such as reduced food consumption or activation of the hypothalamus-pituitary-adrenal axis. Ultimately, the site of action of the cytokine message depends on the localization of cytokine receptors or receptors for intermediates such as prostaglandins E2. These cytokine receptors are difficult to visualize on membranes because the number of receptor sites per cell is very low and they are easily internalized.

Nevertheless, IL-1 receptors were first localized in the granule cell layer of the dentate gyrus, the pyramidal cell layer of the hippocampus and the anterior pituitary gland. More recently, they were identified in endothelial cells of brain venules throughout the brain, at a high density in the preoptic and supraoptic areas of the hypothalamus and the sub-formical organ, and a lower density in the paraventricular hypothalamus, cortex, nucleus of the solitary tract and ventrolateral medulla.

### 14.7. Cytokine Profile in the Brain

Cytokines and chemokines play important roles as mediators of inflammatory reactions in the central nervous system (CNS) and in the process of neuronal-neuroglial interactions that modulate the neuroimmune system. Cytokines may contribute to neuroinflammation as mediators of pro-inflammatory or anti-inflammatory responses within the CNS. Recent studies have been focused on characterizing the profiles of cytokines and chemokines in autistic brains by assessing the relative expression of these proteins in tissue homogenates from medial frontal gyms, anterior cingulate gyms, and cerebellum of autistic and control patients by using cytokine protein array methodology (Huang, R., et al., Methods in Molecular Biology, 278, 215-232). A statistical analysis of the relative expression of cytokines in autistic and control tissues showed a consistent and significantly higher level of subsets of cytokines in the brains of autistic patients. In particular, a larger spectrum of increases in pro-inflammatory and modulatory cytokines was seen in the anterior cingulate gyms, an important cortical structure in autism, where there was a significant increase in pro-inflammatory cytokines such as interleukin-6 (IL-6), interleukin-10 (IL-10), macrophage chemoattractant protein-3 (MCP-3), eotaxin, eotaxin 2, macrophage-derived chemokine (MDC), chemokine-β8 (Ckβ8.1), neutrophil activating peptide-2 (NAP-2), monokine induced by interferon-γ (MIG) and B-lymphocyte chemoattractant (BLC) (Pardo C. et al., International Review of Psychiatry, 2005, 17: 485-495).

The presence of macrophage chemoattractant protein-1 (MCP-1) is of particular interest, since it facilitates the infiltration and accumulation of monocytes and macrophages in inflammatory CNS disease (Mahad, D. and Ransohoff, R., 2003, Seminars in Immunology, 15: 23-32). MCP-1 is produced by activated and reactive astrocytes, a finding that demonstrates the effector role of these cells in the disease process in autism. Studies have suggested that the increase in MCP-1 expression has relevance to the pathogenesis of autism as its elevation in the brain can be linked to pathways of microglial activation and perhaps to the recruitment of monocytes/macrophages to areas of neuronal/cortical abnormalities.

The presence of increased TGF-β1 in the cortex and cerebellum of autistic brains may have important implications for the neurobiology of autism. Transforming growth Factor-β1 is a key anti-inflammatory cytokine and is involved in tissue remodeling following injury. It can suppress specific immune responses by inhibiting T-cell proliferation and maturation and downregulates MHC class II expression (Letterio, J. and Roberts, A., 1998, Annual Review of Immunology, 16, 137-161). Importantly, cells undergoing cell death have been shown to secrete TGF-β1, possibly to reduce local inflammation and prevent degeneration of additional surrounding cells (Chen et al., 2001, Immunity, 14, 715-725). Transforming growth factor-β1 is produced mostly by reactive astrocytes and neurons.

The elevation of TGF-β1 in autistic brains suggests that the elevation of this cytokine in autism may reflect an attempt to modulate neuroinflammation or remodel and repair injured tissue. A profile of cytokine up-regulation was observed in the anterior cingulate gyms, a region in which several cytokines, chemokines, and growth factors were elevated markedly when compared to controls. Pro-inflammatory cytokines (e.g., IL-6) and anti-inflammatory cytokines (e.g., IL-10) as well as subsets of chemokines were elevated in the anterior cingulate gyms, an important cortical region involved in dysfunctional brain activity in autism. These findings support the conclusion that an active, ongoing immunological process was present in multiple areas of the brain but at different levels of expression in each area.

### 15. Autism Spectrum Disorder

Autism Spectrum disorders (ASD) are a heterogeneous group of neurodevelopmental disorders that manifest during early childhood and are characterized by stereotyped interests and impairments in social interaction and communication (American Psychiatric Association, 2000, Diagnostic and Statistical Manual of Mental Disorders DSM-IV-TR, 4^{th} ed. American Psychiatric Association Publishing Inc, Washington D.C.)). Recent epidemiological studies have suggested that ASD is diagnosed in approximately 1% of children (Kogan et al., 2009, Pediatrics, 124(5): 1395-1403). Yet, little is known about the etiology and underlying neuropathology, and there are no clear biological markers for these disorders.

Recent studies have begun to suggest that immune dysfunction is linked in many individuals with ASD, including, marked activation of microglia, increased levels of pro-inflammatory cytokines in brain tissue (Ashwood, P. et al., 2008, J. Neuroimmunol. 204 (1-2), 149-153; Enstrom, A. et al., 2010, Brain Behav. Immun. 24(1): 64-71).

### 16. Schizophrenia

Schizophrenia is a common type of psychosis, characterized by abnormalities in perception, content of thought, and thought processes (hallucinations and delusions) and by extensive withdrawal of interest from other people and the outside world, with excessive focusing on one's own mental life. Schizophrenia has long been associated with immunity, environment and heredity factors (Dantzer R. and Kelley K, Brain Behav. Immun., 2007, 21:153-160; Hart B, Neurosci Biobehav Rev 1988; 12:123-137; Dantzer R. and Kelley K., Life Sci, 1989, 44:1995-2008).

While the exact cause of schizophrenia is not known, several etiological theories have been proposed for the disease, including developmental or neurodegenerative processes, neurotransmitter abnormalities, viral infection, and immune dysfunction or autoimmune mechanisms. In particular, growing evidence suggests that specific neuroimmune and behavioral changes are implicated in the development of schizophrenia. For example, studies have found a relationship between inflammation and schizophrenia, including abnormal cytokines production, abnormal concentrations of cytokines and cytokine receptors in the blood and cerebrospinal fluid in schizophrenia. (Dantzer R., Brain Behav Immun, 2001; 15:7-24; Steptoe, A., Depression and Physical Illness. Cambridge University Press; Cambridge: 2007).

Abnormal regulation of cyto-chemokine activity may contribute to pathophysiology and clinical manifestations in schizophrenic subjects. On the other hand, evidence of reciprocal communication between immune and nervous systems and the altered immunological state in psychiatric diseases have contributed to the “cytokine hypothesis.” On the other hand, cytokines, either directly or indirectly from the periphery, are able to play a role in signaling the brain to produce neurochemical, neuroendocrine, neuroimmune, and behavioral changes. So far the majority of studies in psychiatry have investigated small cyto-chemokine subsets, mainly pro-inflammatory molecules, such as IL-1, IL-6, TNFα, CXCL9, and CXCL11, under various in vitro conditions with peripheral blood preparation, as well as in vivo in various body fluids, such as in plasma, serum, CSF, and urine of patients with schizophrenia (Potvin S. et al., Biological Psychiatry 2008, 63:801-80814; Teixeira A., Prog Neuropsychopharmacol Biol Psychiatry 2008, 32:710-714; Wilke I. et al., European Archives of Psychiatry and Clinical Neuroscience 1996, 246:279-284).

MCP-1 mediates the trans-endothelial migration of inflammatory cells across the blood brain barrier (BBB), modulates the local inflammatory response by forming chemotactic gradients within the CNS and exerts a positive regulatory effect on Th2 cell differentiation by inducing IL-4 (Hayashi M. et al., J. Neuroimmunol., 1995, 60:143-150). IL-8's primary function is the induction of chemotaxis in its target cells. Studies have suggested that circulating levels of IL-8 might be increased in schizophrenic patients (Zhang X. et al., Schizophrenia Research, 2002, 57: 247-258), and high levels of IL-8 have been shown to reduce the chance of good treatment responses to antipsychotic medication in schizophrenia (Zhang X. et al., J. Clin Psychiatry, 2004, 65: 940-947). The importance of IL-8 in schizophrenia is underscored by the finding that patients show increased IL-8 levels, as well as a correlation between these levels and PANSS negative subscale N (Zhang X. et al., Schizophrenia Research, 2002, 57: 247-258). MIP-1α acts by regulating the trafficking and activation state of inflammatory cells, e.g., macrophages, lymphocytes and NK cells, and no different levels of MIP-1α were detected in the cerebrospinal fluid of schizophrenic patients and controls (Nikkila H. et al., Neuropsychobiology, 2002, 46: 169-172). RANTES is thought to promote leukocyte infiltration in sites of inflammation and activate T cells (Schall T. et al., Nature 1990, 347:669-671; Appay V. and Rowland-Jones S., Trends Immunol., 2001, 22: 83-87). IL-18, a member of the IL-1 family, has potent pro-inflammatory properties (Tanaka K. et al., Psychiatry Research 2000, 96: 75-80) and may stimulate the hypothalamicpituitary-adrenal axis and enhance sympathetic nerve system activity, suggesting a pivotal role in psychological processes and psychiatric disorders (Reale M. et al., 2100, BMC Neuroscience, 12:13).

### 17. Free Radicals and N-Acetylcysteine (NAC)

A free radical is a highly reactive and usually short-lived molecular fragment with one or more unpaired electrons. Free radicals are highly chemically reactive molecules. Because a free radical needs to extract a second electron from a neighboring molecule to pair its single electron, it often reacts with other molecules, which initiates the formation of many more free radical species in a self-propagating chain reaction. This ability to be self-propagating makes free radicals highly toxic to living organisms.

Living systems under normal conditions produce the vast majority of free radicals and free radical intermediates. They handle free radicals formed by the breakdown of compounds through the process of metabolism. Most reactive oxygen species come from endogenous sources as by-products of normal and essential metabolic reactions, such as energy generation from mitochondria or the detoxification reactions involving the liver cytochrome P-450 enzyme system. The major sources of free radicals, such as O₂⁻ and HNO₂⁻, are modest leakages from the electron transport chains of mitochondria, chloroplasts, and endoplasmic reticulum.

Reactive oxygen species (“ROS”), such as free radicals and peroxides, represent a class of molecules that are derived from the metabolism of oxygen and exist inherently in all aerobic organisms. The term “oxygen radicals” as used herein refers to any oxygen species that carries an unpaired electron (except free oxygen). The transfer of electrons to oxygen also can lead to the production of toxic free radical species. The best documented of these is the superoxide radical. Oxygen radicals, such as the hydroxyl radical (OH⁻) and the superoxide ion (O2⁻) are very powerful oxidizing agents and cause structural damage to proteins, lipids and nucleic acids. The free radical superoxide anion, a product of normal cellular metabolism, is produced mainly in mitochondria because of incomplete reduction of oxygen. The superoxide radical, although unreactive compared with many other radicals, can be converted by biological systems into other more reactive species, such as peroxyl (ROO⁻), alkoxyl (RO⁻) and hydroxyl (OH⁻) radicals.

The major cellular sources of free radicals under normal physiological conditions are the mitochondria and inflammatory cells, such as granulocytes, macrophages, and some T-lymphocytes, which produce active species of oxygen via the nicotinamide adenine nucleotide oxidase (NADPH oxidase) system, as part of the body's defense against bacterial, fungal or viral infections.

Oxidative injury can lead to widespread biochemical damage within the cell. The molecular mechanisms responsible for this damage are complex. For example, free radicals can damage intracellular macromolecules, such as nucleic acids (e.g., DNA and RNA), proteins, and lipids. Free radical damage to cellular proteins can lead to loss of enzymatic function and cell death. Free radical damage to DNA can cause problems in replication or transcription, leading to cell death or uncontrolled cell growth. Free radical damage to cell membrane lipids can cause the damaged membranes to lose their ability to transport oxygen, nutrients or water to cells.

Biological systems protect themselves against the damaging effects of activated species by several means. These include free radical scavengers and chain reaction terminators; “solid-state” defenses, and enzymes, such as superoxide dismutase, catalase, and the glutathione peroxidase system.

Free radical scavengers/chemical antioxidants, such as vitamin C and vitamin E, counteract and minimize free radical damage by donating or providing unpaired electrons to a free radical and converting it to a nonradical form. Such reducing compounds can terminate radical chain reactions and reduce hydroperoxides and epoxides to less reactive derivatives.

Enzymatic defenses against active free radical species include superoxide dismutase, catalases, and the glutathione reductase/peroxidase system. Superoxide dismutase (SOD) is an enzyme that destroys superoxide radicals. Catalase, a heme-based enzyme which catalyzes the breakdown of hydrogen peroxide into oxygen and water, is found in all living cells, especially in the peroxisomes, which, in animal cells, are involved in the oxidation of fatty acids and the synthesis of cholesterol and bile acids. Hydrogen peroxide is a byproduct of fatty acid oxidation and is produced by white blood cells to kill bacteria.

Glutathione, a tripeptide composed of glycine, glutamic acid, and cysteine that contains a nucleophilic thiol group, is widely distributed in animal and plant tissues. It exists in both the reduced thiol form (GSH) and the oxidized disulfide form (GSSG). In its reduced GSH form, glutathione acts as a substrate for the enzymes GSH-S-transferase and GSH peroxidase, both of which catalyze reactions for the detoxification of xenobiotic compounds, and for the antioxidation of reactive oxygen species and other free radicals. Glutathione detoxifies many highly reactive intermediates produced by cytochrome P450 enzymes in phase I metabolism. Without adequate GSH, the reactive toxic metabolites produced by cytochrome P-450 enzymes may accumulate causing organ damage.

Glutathione (GSH) plays key roles in cellular metabolism and protection against oxidative and other toxic molecules, including those generated in response to attack by cytokines that induce pain and fever. Stores of reduced GSH are influenced greatly by nutritional status, presence of certain disease states, and exposures to oxidative stressors and molecules that are detoxified by conjugation with GSH. Viral, bacterial, and fungal infections, malnutrition, chronic and acute alcohol consumption, diabetes, certain metabolic diseases, and consumption of oxidative drugs all have been shown to decrease GSH.

Decreased levels of GSH are known to be associated with increased pain and fever while increased GSH levels are known to be associated with decreased pain and fever. Consistent with this inverse relationship between GSH levels and signs of inflammation (pain and/or fever), decreasing GSH renders cells more sensitive to the effects of cytokines (e.g., IL-1, IL-6, and TNF) that increase inflammation, pain and fever whereas NAC administration, which acts primarily to restore GSH, is known to decrease levels of IL-1, IL-6, and TNF and to reduce fever and pain.

TABLE 4
Diseases in which GSH Deficiency Has Been Demonstrated.

| Classification | Disease |
|---|---|
| Hepatic Function | Acetaminophen toxicity |
| | Alcoholism |
| | Hepatitis |
| Renal Function | Chronic Kidney Failure |
| | Dialysis |
| | Nephrotoxicity |
| | Alpha-Amanitin poisoning |
| Cardiovascular | Angina |
| | Arteriosclerosis/Cardiac Risk |
| | Myocardiac Infarction |
| | Cardiomyopathy |
| Endocrine | Diabetes |
| Pulmonary | Bronchopulmonary |
| | Acute Respiratory Distress Syndrome |
| | (ARDS) |
| | Fibrosing Alveolitis |
| | Chronic Asthma |
| | Chronic Bronchitis/Chronic |
| | Obstructive Pulmonary Disease |
| | (COPD) |
| | Cystic Fibrosis |
| | Pulmonary Fibrosis |
| | Smoking |
| | Lung Cancer |
| Critical Care | Intensive Care |
| | Sepsis/Septic Shock |
| | Malnutrition |
| | Epilepsy |
| Infection | HIV |
| | Helicobacter pylori |
| | Influenza |
| | Malaria |
| Gastrointestinal | Inflammatory Bowel Disease |
| | Barrett's Esophagus |
| | Liver Disease |
| | Liver Transplantation |
| | Colon Cancer |
| Optic | Blepharitis |
| | Cataract |
| | Eale's Disease |
| Skin | Psoriasis |
| | Photodermatitis |
| Immune system | Rheumatoid Arthritis |
| | Common Variable Immunodeficiency |
| Urogenital | Prostate |
| | Urinary |
| Muscular | Exercise |
| Aging | |
| Toxic Agents | Arsenic Poisoning |
| | Other Chemicals and Medications |
| Perinatal | Preeclampsia |
| | Neonates |
| Metabolism | Phenylketonuria |

Glutathione reductase (NADPH), a flavoprotein enzyme of the oxidoreductase class, is essential for the maintenance of cellular glutathione in its reduced form (Carlberg & Mannervick, J. Biol. Chem. 250: 5475-80 (1975)). It catalyzes the reduction of oxidized glutathione (GSSG) to reduced glutathione (GSH) in the presence of NADPH and maintains a high intracellular GSH/GSSG ratio of about 500:1 in red blood cells.

Synthesis of GSH requires cysteine, a conditionally essential amino acid that must be obtained from dietary sources or by conversion of dietary methionine via the cystathionase pathway. If the supply of cysteine is adequate, normal GSH levels are maintained. But GSH depletion occurs if supplies of cysteine are inadequate to maintain GSH homeostasis in the face of increased GSH consumption. Acute GSH depletion causes severe—often fatal—oxidative and/or alkylation injury, and chronic or slow arising GSH deficiency due to administration of GSH-depleting drugs, such as acetaminophen, or to diseases and conditions that deplete GSH, can be similarly debilitating.

Replenishment of GSH requires an exogenous thiol supply, which usually is acquired by ingestion of cysteine or methionine in protein or other form. It also can be acquired by ingestion of NAC, a cysteine prodrug that is administered as the standard treatment for GSH deficiency. When administered orally or intravenously, NAC is rapidly converted to cysteine, which is then converted to GSH in the liver and elsewhere by highly regulated conversion mechanisms that maintain optimal levels of reduced GSH as long as sufficient cysteine is available for the purpose.

Cysteine is necessary to replenish hepatocellular GSH. Although various forms of cysteine and its precursors have been used as nutritional and therapeutic sources of cysteine, N-acetylcysteine (NAC) is the most widely used and extensively studied. NAC is about 10 times more stable than cysteine and much more soluble than the stable cysteine disulfide, cystine.

Glutathione, glutathione monoethyl ester, and L-2-oxothiazolidine-4-carboxylate (procysteine/OTC) also have been used effectively in some studies. In addition, dietary methionine and S-adenosylmethionine are an effective source of cysteine.

Besides NAC's scavenger function, it is well-known that NAC promotes cellular glutathione production, and thus reduces, or even prevents, oxidant mediated damage. Indeed, treatment with NAC provides beneficial effects in a number of respiratory, cardiovascular, endocrine, infectious, and other disease settings as described in W005/017094, which is incorporated by reference herein. For example, rapid administration of NAC is the standard of care for preventing hepatic injury in acetaminophen overdose. NAC administered intravenously in dogs has been shown to protect against pulmonary oxygen toxicity and against ischemic and reperfusion damage (Gillissen, A., and Nowak, A., Respir. Med. 92: 609-23, 613 (1998)). NAC also has anti-inflammatory properties.

18. Investigations into Platelet Release

While inflammatory chemokines have been suggested to be expressed and secreted into the serum in some diseases, a therapeutic correlation between the level of an inflammatory chemokine in a blood sample and the status or progress of a specific disease in a subject has not been established. Moreover, the prior attempts to identify such correlation in the blood sample of a patient using conventional fractionation methods have been hampered by contamination of the sample with platelets or platelet-derived factors during the fractionation process.

The described invention provides a method for correlating expression levels of specific inflammatory chemokines therapeutically with the status or progress of a platelet-dependent disease. The described invention further provides methods that can reduce the contamination of a blood sample by platelets and platelet-derived factors during fractionation, which ultimately allows effective and efficient monitoring of inflammatory cytokine levels and evaluation of the therapeutic efficacy of a drug in the treatment of platelet-dependent diseases.

### SUMMARY

According to one aspect, the described invention provides a method for treating a disease, disorder or condition comprising an inflammatory component that includes platelet dysfunction comprising: (a) obtaining a whole blood sample from a subject with the disease or disorder, wherein the whole blood sample comprises blood cells and nonactivated platelets; (b) purifying the blood sample to yield a purified blood sample substantially free of the blood cells and the nonactivated platelets; (c) measuring an amount of at least one marker for platelet dysfunction in the purified blood sample of (b); (e) comparing the amount of the marker for platelet dysfunction in the purified blood sample measured in (c) with the amount of the at least one marker for platelet dysfunction in a control blood sample; wherein an increased amount of the marker for platelet dysfunction in the purified blood sample compared to the amount of the at least one marker in the control blood sample indicates that the subject is susceptible to treatment with the treatment regimen; and (f) after determining that the subject is susceptible to treatment with the treatment regimen, implementing the treatment regimen comprising administering a composition comprising a therapeutic amount of N-acetylcysteine or a derivative of N-acetylcysteine containing one or more functional groups selected from the group consisting of an aliphatic group, an aromatic group, a heterocyclic radical group, an epoxide group, and an arene oxide group, and a pharmaceutically acceptable carrier, wherein the therapeutic amount is effective to decrease the inflammation due to platelet dysfunction. According to one embodiment of the method, the at least one marker for platelet dysfunction is an inflammatory chemokine. According to another embodiment, the inflammatory chemokine is Regulated upon Activation, Normal T-cell Expressed and Secreted (RANTES/CCL-5). According to another embodiment, the inflammatory chemokine is Platelet Factor-4 (CXCL-4/PF-4). According to another embodiment, measuring step (c) is carried out by a fluid-based assay. According to another embodiment, the fluid-based assay comprises an enzyme-linked immunosorbent assay (ELISA), bead-based immunoassay, mass spectrometry, nuclear magnetic resonance spectroscopy, and a combination thereof. According to another embodiment, the disease or disorder is a mucosal disease. According to another embodiment, the mucosal disease is cystic fibrosis. According to another embodiment, the disease or disorder is a nervous system disease. According to another embodiment, the nervous system disease is autism. According to another embodiment, the nervous system disease is an autism spectrum disorder. According to another embodiment, the nervous system disease is schizophrenia.

According to another aspect, the described invention provides a method for managing a disease comprising an inflammatory component that includes platelet dysfunction comprising (a) monitoring therapeutic efficacy of a treatment regimen for treating the disease in a subject, wherein the treatment regimen comprises administering a dose of a pharmaceutical composition comprising a therapeutic amount of N-acetylcysteine and a pharmaceutically acceptable carrier, by: (1) obtaining a control whole blood sample from the subject prior to initiating the therapeutic regimen and at least one test whole blood sample from the subject after administering the dose of the pharmaceutical composition, wherein each of the control and test whole blood samples comprise blood cells and nonactivated platelets; (2) purifying the control and test whole blood samples to yield to yield a control purified blood sample and a test purified blood sample wherein each of the control purified blood sample and the test purified blood sample is substantially free of the blood cells and the nonactivated platelets; (3) measuring an amount of at least one marker for platelet dysfunction in the control purified blood sample and in the test purified blood sample; and (4) comparing the amount of the marker for platelet dysfunction in the control purified control blood sample with the amount of the marker for platelet dysfunction in the test purified test blood sample; wherein a decreased amount of the marker for platelet dysfunction in the test purified test blood sample compared to the amount of the marker for platelet dysfunction in the control purified blood sample indicates that the pharmaceutical composition comprising a therapeutic amount of N-acetylcysteine or a derivative of N-acetylcysteine containing one or more functional groups selected from the group consisting of an aliphatic group, an aromatic group, a heterocyclic radical group, an epoxide group, and an arene oxide group and a pharmaceutically acceptable carrier remains effective for treating the platelet dysfunction and (b) continuing the treatment regimen. According to one embodiment of the method, the disease comprising platelet dysfunction is characterized by an elevated level of Regulated upon Activation, Normal T-cell Expressed and Secreted (RANTES/CCL-5) or Platelet Factor-4 (CXCL-4/PF-4). According to another embodiment, the marker for platelet dysfunction is an inflammatory chemokine. According to another embodiment, the inflammatory chemokine is Regulated upon Activation, Normal T-cell Expressed and Secreted (RANTES/CCL-5). According to another embodiment, the inflammatory chemokine is Platelet Factor-4 (CXCL-4/PF-4). According to another embodiment, measuring step (c) is carried out by a fluid-based assay. According to another embodiment, the fluid-based assay comprises an enzyme-linked immunosorbent assay (ELISA), bead-based assay (such as, cytometric bead array or Luminex-type assay), mass spectrometry, and nuclear magnetic resonance. According to another embodiment, the platelet-dependent disease is a mucosal disease. According to another embodiment, the mucosal disease is cystic fibrosis. According to another embodiment, the platelet-dependent disease is a nervous system disease. According to another embodiment, the nervous system disease is autism. According to another embodiment, the nervous system disease is an autism spectrum disorder. According to another embodiment, the nervous system disease is schizophrenia.

### BRIEF DESCRIPTION OF THE DRAWINGS

FIG. 1 shows the mechanism of thrombosis

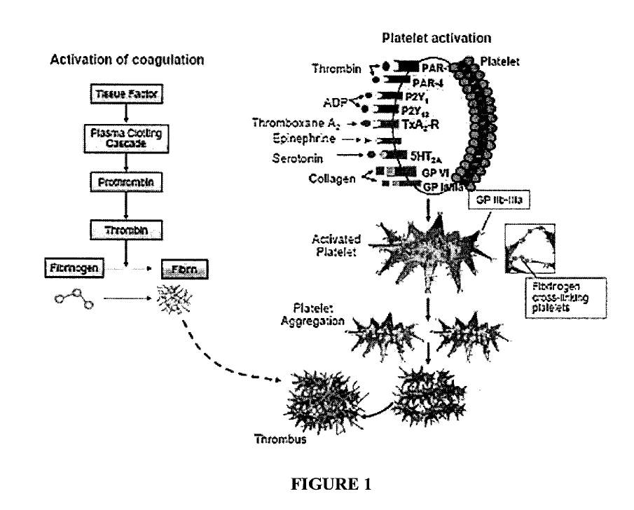

FIG. 2 shows inside-out signaling of platelet agonists

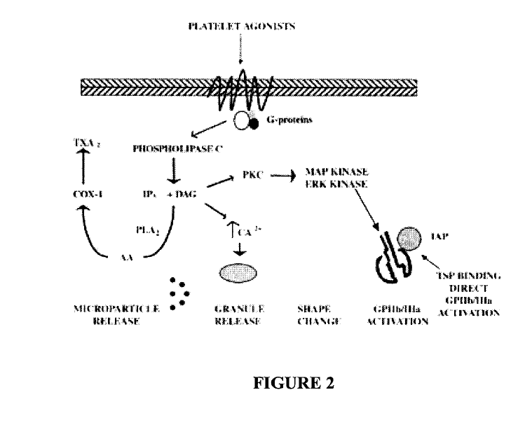

FIG. 3 shows the neural pathway (a) and the humoral pathway, which transduce immune signals from the periphery to the brain. The abbreviations are as follows: (a) NTS, nucleus tractus solitarius; PB, parabrachial nucleus; VLM, ventrolateral medulla; PVN, hypothalamic paraventricular nuclei; SON supraoptic nuclei; CEA, central amygdala; BNST, bed nucleus of the stria sterminalis, PAG, periaqueductal grey (b) CP, choroid plexus; ME, median eminence; OVLT, organum vasculosum of the laminae terminalis; AP, area postrema, SFO, supraformical organ; TLR, toll-like receptor; BBB, and blood-brain barrier

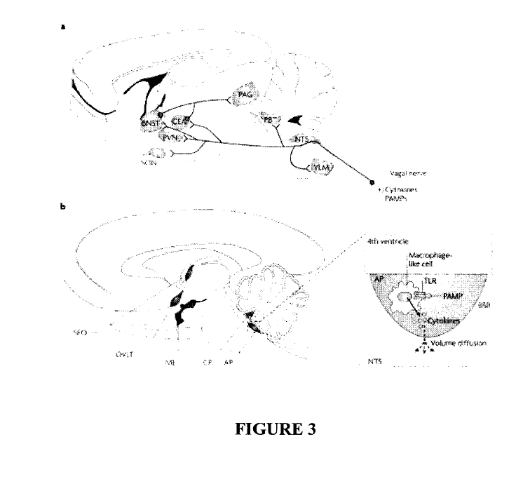

### DETAILED DESCRIPTION OF THE INVENTION

### Glossary

The term “administer” as used herein means to give or to apply. The term “administering” as used herein includes in vivo administration, as well as administration directly to tissue ex vivo. Generally, compositions may be administered systemically either orally, buccally, parenterally, topically, by inhalation or insufflation (i.e., through the mouth or through the nose), or rectally in dosage unit formulations containing conventional nontoxic pharmaceutically acceptable carriers, adjuvants, and vehicles as desired, or may be locally administered by means such as, but not limited to, injection, implantation, grafting, topical application, or parenterally.

The term “antagonist” as used herein refers to a molecule or substance, which decreases the amount or the duration of the effect of the biological activity of another molecule or substance. Antagonists may include proteins, nucleic acids, carbohydrates, antibodies or any other molecules, which decrease the effect of another molecule substance.

As used herein, the term “antibody” includes, by way of example, both naturally occurring and non-naturally occurring antibodies. Specifically, the term “antibody” includes polyclonal antibodies and monoclonal antibodies, and fragments thereof. Furthermore, the term “antibody” includes chimeric antibodies and wholly synthetic antibodies, and fragments thereof.

Antibodies are serum proteins the molecules of which possess small areas of their surface that are complementary to small chemical groupings on their targets. These complementary regions (referred to as the antibody combining sites or antigen binding sites) of which there are at least two per antibody molecule, and in some types of antibody molecules ten, eight, or in some species as many as 12, may react with their corresponding complementary region on the antigen (the antigenic determinant or epitope) to link several molecules of multivalent antigen together to form a lattice.

The basic structural unit of a whole antibody molecule consists of four polypeptide chains, two identical light (L) chains (each containing about 220 amino acids) and two identical heavy (H) chains (each usually containing about 440 amino acids). The two heavy chains and two light chains are held together by a combination of noncovalent and covalent (disulfide) bonds. The molecule is composed of two identical halves, each with an identical antigen-binding site composed of the N-terminal region of a light chain and the N-terminal region of a heavy chain. Both light and heavy chains usually cooperate to form the antigen binding surface.

Human antibodies show two kinds of light chains, κ and λ; individual molecules of immunoglobulin generally are only one or the other. In normal serum, 60% of the molecules have been found to have κ determinants and 30 percent λ. Many other species have been found to show two kinds of light chains, but their proportions vary. For example, in the mouse and rat, λ chains comprise but a few percent of the total; in the dog and cat, κ chains are very low; the horse does not appear to have any κ chain; rabbits may have 5 to 40% λ, depending on strain and b-locus allotype; and chicken light chains are more homologous to λ than κ.

In mammals, there are five classes of antibodies, IgA, IgD, IgE, IgG, and IgM, each with its own class of heavy chain-α (for IgA), δ (for IgD), ε (for IgE), γ (for IgG) and μ (for IgM). In addition, there are four subclasses of IgG immunoglobulins (IgG1, IgG2, IgG3, IgG4) having γ1, γ2, γ3, and γ4 heavy chains respectively. In its secreted form, IgM is a pentamer composed of five four-chain units, giving it a total of 10 antigen binding sites. Each pentamer contains one copy of a J chain, which is covalently inserted between two adjacent tail regions.

All five immunoglobulin classes differ from other serum proteins in that they show a broad range of electrophoretic mobility and are not homogeneous. This heterogeneity—that individual IgG molecules, for example, differ from one another in net charge—is an intrinsic property of the immunoglobulins.

The principle of complementarity, which often is compared to the fitting of a key in a lock, involves relatively weak binding forces (hydrophobic and hydrogen bonds, van der Waals forces, and ionic interactions), which are able to act effectively only when the two reacting molecules can approach very closely to each other and indeed so closely that the projecting constituent atoms or groups of atoms of one molecule can fit into complementary depressions or recesses in the other. Antigen-antibody interactions show a high degree of specificity, which is manifest at many levels. Brought down to the molecular level, specificity means that the combining sites of antibodies to an antigen have a complementarity not at all similar to the antigenic determinants of an unrelated antigen. Whenever antigenic determinants of two different antigens have some structural similarity, some degree of fitting of one determinant into the combining site of some antibodies to the other may occur, and that this phenomenon gives rise to cross-reactions. Cross reactions are of major importance in understanding the complementarity or specificity of antigen-antibody reactions. Immunological specificity or complementarity makes possible the detection of small amounts of impurities/contaminations among antigens.

Monoclonal antibodies (mAbs) can be generated by fusing mouse spleen cells from an immunized donor with a mouse myeloma cell line to yield established mouse hybridoma clones that grow in selective media. A hybridoma cell is an immortalized hybrid cell resulting from the in vitro fusion of an antibody-secreting B cell with a myeloma cell. In vitro immunization, which refers to primary activation of antigen-specific B cells in culture, is another well-established means of producing mouse monoclonal antibodies.

Diverse libraries of immunoglobulin heavy (VH) and light (Vκ and Vλ) chain variable genes from peripheral blood lymphocytes also can be amplified by polymerase chain reaction (PCR) amplification. Genes encoding single polypeptide chains in which the heavy and light chain variable domains are linked by a polypeptide spacer (single chain Fv or scFv) can be made by randomly combining heavy and light chain V-genes using PCR. A combinatorial library then can be cloned for display on the surface of filamentous bacteriophage by fusion to a minor coat protein at the tip of the phage.

The technique of guided selection is based on human immunoglobulin V gene shuffling with rodent immunoglobulin V genes. The method entails (i) shuffling a repertoire of human λ light chains with the heavy chain variable region (VH) domain of a mouse monoclonal antibody reactive with an antigen of interest; (ii) selecting half-human Fabs on that antigen (iii) using the selected λ light chain genes as “docking domains” for a library of human heavy chains in a second shuffle to isolate clone Fab fragments having human light chain genes; (v) transfecting mouse myeloma cells by electroporation with mammalian cell expression vectors containing the genes; and (vi) expressing the V genes of the Fab reactive with the antigen as a complete IgG1, λ antibody molecule in the mouse myeloma.

The term “antigen” and its various grammatical forms refers to any substance that can stimulate the production of antibodies and can combine specifically with them. The terms “epitope” and “antigenic determinant” are used interchangeably herein to refer to an antigenic site on a molecule that an antibody combining site (ACS) recognizes and to which that antibody binds/attaches itself. A given epitope may be primary, secondary, or tertiary-sequence related. Sequential antigenic determinants/epitopes essentially are linear chains. In ordered structures, such as helical polymers or proteins, the antigenic determinants/epitopes essentially would be limited regions or patches in or on the surface of the structure involving amino acid side chains from different portions of the molecule which could come close to one another. These are conformational determinants.

As used herein the term “blood” refers to whole blood, processed blood, venous blood, arterial blood, blood from bone-marrow, umbilical cord blood, and placenta blood.

The term “whole blood” as used herein refers to generally unprocessed or unmodified collected blood containing all of its components, including, but are not limited to, plasma, cellular components (e.g., red blood cells, white blood cells (including lymphocytes, monocytes, eosinophils, basophils, and neutrophils), platelets, proteins (e.g., fibrinogen, albumin, immunoglobulins), hormones, coagulation factors, and fibrinolytic factors. The term “whole blood” is inclusive of any anticoagulant that may be combined with the blood upon collection.

As used herein the term “blood component” refers to erythrocytes (red blood cells), leuckocytes (white blood cells), monocytes, platelets, fibrinogen, and thrombin.

The term “blood-brain barrier” as used herein refers to a series of structures that limit the penetration and diffusion of circulating water-soluble substances into the brain and include tight junctions between endothelial cells of brain capillaries, a dense network of astrocytes, a reduced volume of extracellular milieu and efflux pumps.

The term “carrier” as used herein describes a material that does not cause significant irritation to an organism and does not abrogate the biological activity and properties of the compound of the composition of the described invention. Carriers must be of sufficiently high purity and of sufficiently low toxicity to render them suitable for administration to the mammal being treated. The carrier can be inert, or it can possess pharmaceutical benefits. The terms “excipient”, “carrier”, or “vehicle” are used interchangeably to refer to carrier materials suitable for formulation and administration of pharmaceutically acceptable compositions described herein. Carriers and vehicles useful herein include any such materials know in the art which are nontoxic and do not interact with other components.

The term “centrifuge” or “centrifugation” as used herein refers to techniques for separating contained materials of different specific gravities, or to separate colloidal particles suspended in a liquid. The term “centrifuge” as used herein also refers to any container that has a rotating rotor, rotating screw or other rotating part that provides a centrifugal force.

The term “chemokine” as used herein refers to a class of chemotactic cytokines that signal leukocytes to move in a specific direction.

The terms “chemotaxis” or “chemotactic” refer to the directed motion of a motile cell or part along a chemical concentration gradient towards environmental conditions it deems attractive and/or away from surroundings it finds repellent.

The term “choroid plexus” as used herein refers to a capillary bed that is covered by transporting ependymal cells and that protrudes into the cerebral ventricles. The ependymal cells are responsible for producing cerebral spinal fluid.

The term “component” as used herein refers to a constituent part, element or ingredient.

The term “condition”, as used herein, refers to a variety of health states and is meant to include disorders or diseases caused by any underlying mechanism or disorder, injury, and the promotion of healthy tissues and organs.

The term “contact” and all its grammatical forms as used herein refers to a state or condition of touching or of immediate or local proximity

The term “cytokine” as used herein refers to small soluble protein substances secreted by cells which have a variety of effects on other cells. Cytokines mediate many important physiological functions including growth, development, wound healing, and the immune response. They act by binding to their cell-specific receptors located in the cell membrane, which allows a distinct signal transduction cascade to start in the cell, which eventually will lead to biochemical and phenotypic changes in target cells. Generally, cytokines act locally. They include type I cytokines, which encompass many of the interleukins, as well as several hematopoietic growth factors; type II cytokines, including the interferons and interleukin-10; tumor necrosis factor (“TNF”)-related molecules, including TNF-α and lymphotoxin; immunoglobulin super-family members, including interleukin 1 (“IL-1”); and the chemokines, a family of molecules that play a critical role in a wide variety of immune and inflammatory functions. The same cytokine can have different effects on a cell depending on the state of the cell. Cytokines often regulate the expression of, and trigger cascades of, other cytokines.

The term “inflammatory cytokines” or “inflammatory mediators” as used herein refers to the molecular mediators of the inflammatory process. These soluble, diffusible molecules act both locally at the site of tissue damage and infection and at more distant sites. Some inflammatory mediators are activated by the inflammatory process, while others are synthesized and/or released from cellular sources in response to acute inflammation or by other soluble inflammatory mediators. Examples of inflammatory mediators of the inflammatory response include, but are not limited to, plasma proteases, complement, kinins, clotting and fibrinolytic proteins, lipid mediators, prostaglandins, leukotrienes, platelet-activating factor (PAF), peptides and amines, including, but not limited to, histamine, serotonin, and neuropeptides, proinflammatory cytokines, including, but not limited to, interleukin-1-beta (IL-1β), interleukin-4 (IL-4), interleukin-6 (IL-6), interleukin-8 (IL-8), tumor necrosis factor-alpha (TNF-α), interferon-gamma (IF-γ), and interleukin-12 (IL-12),

Among the pro-inflammatory mediators, IL-1, IL-6, and TNF-α are known to activate hepatocytes in an acute phase response to synthesize acute-phase proteins that activate complement. Complement is a system of plasma proteins that interact with pathogens to mark them for destruction by phagocytes. Complement proteins can be activated directly by pathogens or indirectly by pathogen-bound antibody, leading to a cascade of reactions that occurs on the surface of pathogens and generates active components with various effector functions. IL-1, IL-6, and TNF-α also activate bone marrow endothelium to mobilize neutrophils, and function as endogenous pyrogens, raising body temperature, which helps eliminating infections from the body. A major effect of the cytokines is to act on the hypothalamus, altering the body's temperature regulation, and on muscle and fat cells, stimulating the catabolism of the muscle and fat cells to elevate body temperature. At elevated temperatures, bacterial and viral replication are decreased, while the adaptive immune system operates more efficiently.

The term “inflammatory chemokine” as used herein refers to a chemotactic cytokine, which is induced in response to inflammatory stimuli. Inflammatory chemokines attract inflammatory cells to site of inflammation. Examples of inflammatory chemokines include, but are not limited to, monocyte chemotactic protein-1 (MCP-1/CCL2), macrophage inflammatory protein 1 alpha (MIP-1α/CCL3), macrophage inflammatory protein 1 beta (MIP-1β/CCL4), Regulated upon Activation, Normal T cell Expressed and Secreted (RANTES/CCL5), platelet factor-4 (CXCL4/PF-4).

The term “tumor necrosis factor” as used herein refers to a cytokine made by white blood cells in response to an antigen or infection, which induce necrosis (death) of tumor cells and possesses a wide range of pro-inflammatory actions. Tumor necrosis factor also is a multifunctional cytokine with effects on lipid metabolism, coagulation, insulin resistance, and the function of endothelial cells lining blood vessels.

The term “interleukin (IL)” as used herein refers to a cytokine secreted by, and acting on, leukocytes. Interleukins regulate cell growth, differentiation, and motility, and stimulates immune responses, such as inflammation. Examples of interleukins include, interleukin-1 (IL-1), interleukin-1β (IL-1β), interleukin-6 (IL-6), interleukin-8 (IL-8), and interleukin-12 (IL-12).

The term “derivative” as used herein refers to a compound that may be produced from another compound of similar structure in one or more steps. A “derivative” or “derivatives” of a compound retains at least a degree of the desired function of the compound. Accordingly, an alternate term for “derivative” may be “functional derivative.”

The derivatives of N-acetylcysteine, for example, contain one or more functional groups (e.g., aliphatic, aromatic, heterocyclic radicals, epoxides, and/or arene oxides) incorporated into N-acetylcysteine. According to another embodiment, the derivatives of N-acetylcysteine disclosed herein also comprise “prodrugs” of N-acetylcysteine, which are either active in the prodrug form or are cleaved in vivo to the parent active compound. According to another embodiment, the derivatives of N-acetylcysteine also includes any pharmaceutically acceptable salt, ester, solvate, hydrate or any other compound, which, upon administration to the recipient, is capable of providing (directly or indirectly) N-acetylcysteine.

As used herein the term “diagnose” refers to the act or process of identifying or determining a disease or condition in a mammal or the cause of a disease or condition by the evaluation of the signs and symptoms of the disease or disorder.

The term “disease” or “disorder”, as used herein, refers to an impairment of health or a condition of abnormal functioning.

The term “drug” as used herein refers to a therapeutic agent or any substance, other than food, used in the prevention, diagnosis, alleviation, treatment, or cure of disease.

The term “dye” (also referred to as “fluorochrome” or “fluorophore”) as used herein refers to a component of a molecule which causes the molecule to be fluorescent. The component is a functional group in the molecule that absorbs energy of a specific wavelength and re-emits energy at a different (but equally specific) wavelength. The amount and wavelength of the emitted energy depend on both the dye and the chemical environment of the dye. Many dyes are known, including, but not limited to, FITC, R-phycoerythrin (PE), PE-Texas Red Tandem, PE-Cy5 Tandem, propidium iodem, EGFP, EYGP, ECF, DsRed, allophycocyanin (APC), PerCp, SYTOX Green, courmarin, Alexa Fluors (350, 430, 488, 532, 546, 555, 568, 594, 633, 647, 660, 680, 700, 750), Cy2, Cy3, Cy3.5, Cy5, Cy5.5, Cy7, Hoechst 33342, DAPI, Hoechst 33258, SYTOX Blue, chromomycin A3, mithramycin, YOYO-1, SYTOX Orange, ethidium bromide, 7-AAD, acridine orange, TOTO-1, TO-PRO-1, thiazole orange, TOTO-3, TO-PRO-3, thiazole orange, propidium iodide (PI), LDS 751, Indo-1, Fluo-3, DCFH, DHR, SNARF, Y66F, Y66H, EBFP, GFPuv, ECFP, GFP, AmCyanl, Y77W, S65A, S65C, S65L, S65T, ZsGreen1, ZsYellow1, DsRed2, DsRed monomer, AsRed2, mRFP1, HcRed1, monochlorobimane, calcein, the DyLight Fluors, cyanine, hydroxycoumarin, aminocoumarin, methoxycoumarin, Cascade Blue, Lucifer Yellow, NBD, PE-Cy5 conjugates, PE-Cy7 conjugates, APC-Cy7 conjugates, Red 613, fluorescein, FluorX, BODIDY-FL, TRITC, Xrhodamine, Lissamine Rhodamine B, Texas Red, TruRed, and derivatives thereof.

The term “prodrug” as used herein means a derivative, which is in an inactive form but is converted to an active form by biological conversion following administration to a subject.

The term “effective amount” refers to the amount necessary or sufficient to realize a desired biologic effect.

The term “eosinophils” or “eosinophil granulocytes” as used herein refers to white blood cells responsible for combating multicellular parasites and certain infections in vertebrates. They are granulocytes that develop during hematopoiesis in the bone marrow before migrating into blood. Along with mast cells, they also control mechanisms associated with allergy and asthma. Following activation, eosinophils exert diverse functions, including (1) production of cationic granule proteins and their release by degranulation, (2) production of reactive oxygen species, such as, superoxide, peroxide, and hypobromite (hypobromous acid, which is preferentially produced by eosinophil peroxidase), (3) production of lipid mediators, such as, eicosanoids from leukotriene and prostaglandin families, (4) production of growth factors, such as transforming growth factor (TGF-β), vascular endothelial growth factor (VEGF), and platelet-derived growth factor (PDGF), and (5) production of cytokines such as IL-1, IL-2, IL-4, IL-5, IL-6, IL-8, IL-13, and TNF-α.

The term “fibrosis” as used herein refers to the formation or development of excess fibrous connective tissue in an organ or tissue as a result of injury or inflammation of a part, or of interference with its blood supply. It may be a consequence of the normal healing response leading to a scar, or it may be an abnormal, reactive process.

The term “flow cytometry” as used herein refers to a tool for interrogating the phenotype and characteristics of cells. Flow cytometry is a system for sensing cells or particles as they move in a liquid stream through a laser (light amplification by stimulated emission of radiation)/light beam past a sensing area. The relative light-scattering and color-discriminated fluorescence of the microscopic particles is measured Analysis and differentiation of the cells is based on size, granularity, and whether the cells is carrying fluorescent molecules in the form of either antibodies or dyes. As the cell passes through the laser beam, light is scattered in all directions, and the light scattered in the forward direction at low angles (0.5-10°) from the axis is proportional to the square of the radius of a sphere and so to the size of the cell or particle. Light may enter the cell; thus, the 90° light (right-angled, side) scatter may be labled with fluorochrome-linked antibodies or stained with fluorescent membrane, cytoplasmic, or nuclear dyes. Thus, the differentiation of cell types, the presence of membrane receptors and antigens, membrane potential, PH, enzyme activity, and DNA content may be facilitated. Flow cytometers are multiparameter, recording several measurements on each cell; therefore, it is possible to identify a homogeneous subpopulation within a heterogeneous population (Marion G. Macey, Flow cytometry: principles and applications, Humana Press, 2007).

The term “fluorescence” as used herein refers to the result of a three-state process that occurs in certain molecules, generally referred to as “fluorophores” or “fluorescent dyes,” when a molecule or nanostructure relaxes to its ground state after being electrically excited. Stage 1 involves the excitation of a fluorophore through the absorption of light energy; Stage 2 involves a transient excited lifetime with some loss of energy; and Stage 3 involves the return of the fluorophore to its ground state accompanied by the emission of light.

The term “fluorescent-activated cell sorting” (also referred to as “FACS”) as used herein refers to a method for sorting a heterogeneous mixture of biological cells into one or more containers, one cell at a time, based upon the specific light scattering and fluorescent characteristics of each cell.

The term “fractionate” and its various grammatical forms as used herein refers to separating or dividing into component parts, fragments, or divisions.

The term “hemostasis” as used herein refers to cessation of flow of blood through a partial or complete defect in a blood vessel wall.

The term “inflammation” as used herein refers to the physiologic process by which vascularized tissues respond to injury. See, e.g., FUNDAMENTAL IMMUNOLOGY, 4th Ed., William E. Paul, ed. Lippincott-Raven Publishers, Philadelphia (1999) at 1051-1053, incorporated herein by reference. During the inflammatory process, cells involved in detoxification and repair are mobilized to the compromised site by inflammatory mediators. Inflammation is often characterized by a strong infiltration of leukocytes at the site of inflammation, particularly neutrophils (polymorphonuclear cells). These cells promote tissue damage by releasing toxic substances at the vascular wall or in uninjured tissue. Traditionally, inflammation has been divided into acute and chronic responses.

The term “acute inflammation” as used herein refers to the rapid, short-lived (minutes to days), relatively uniform response to acute injury characterized by accumulations of fluid, plasma proteins, and neutrophilic leukocytes. Examples of injurious agents that cause acute inflammation include, but are not limited to, pathogens (e.g., bacteria, viruses, parasites), foreign bodies from exogenous (e.g. asbestos) or endogenous (e.g., urate crystals, immune complexes), sources, and physical (e.g., burns) or chemical (e.g., caustics) agents.

The term “chronic inflammation” as used herein refers to inflammation that is of longer duration and which has a vague and indefinite termination. Chronic inflammation takes over when acute inflammation persists, either through incomplete clearance of the initial inflammatory agent or as a result of multiple acute events occurring in the same location. Chronic inflammation, which includes the influx of lymphocytes and macrophages and fibroblast growth, may result in tissue scarring at sites of prolonged or repeated inflammatory activity.

The terms “inhibiting”, “inhibit” or “inhibition” are used herein to refer to reducing the amount or rate of a process, to stopping the process entirely, or to decreasing, limiting, or blocking the action or function thereof. Inhibition may include a reduction or decrease of the amount, rate, action function, or process of a substance by at least 5%, at least 10%, at least 15%, at least 20%, at least 25%, at least 30%, at least 40%, at least 45%, at least 50%, at least 55%, at least 60%, at least 65%, at least 70%, at least 75%, at least 80%, at least 85%, at least 90%, at least 95%, at least 98%, or at least 99%.

The term “inhibitor” as used herein refers to a second molecule that binds to a first molecule thereby decreasing the first molecule's activity. In the context of an enzyme, for example, enzyme inhibitors are molecules that bind to enzymes thereby decreasing enzyme activity. The binding of an inhibitor may stop substrate from entering the active site of the enzyme and/or hinder the enzyme from catalyzing its reaction Inhibitor binding is either reversible or irreversible. Irreversible inhibitors usually react with the enzyme and change it chemically, for example, by modifying key amino acid residues needed for enzymatic activity. In contrast, reversible inhibitors bind non-covalently and produce different types of inhibition depending on whether these inhibitors bind the enzyme, the enzyme-substrate complex, or both. Enzyme inhibitors often are evaluated by their specificity and potency.

The term “leukocyte” or “white blood cell (WBC)” as used herein refers to a type of immune cell. Most leukocytes are made in the bone marrow and are found in the blood and lymph tissue. Leukocytes help the body fight infections and other diseases. Granulocytes, monocytes, and lymphocytes are leukocytes.

The term “macrophage” as used herein refers to a type of white blood cell that surrounds and kills microorganisms, removes dead cells, and stimulates the action of other immune system cells. After digesting a pathogen, a macrophage presents an antigen (a molecule, most often a protein found on the surface of the pathogen, used by the immune system for identification) of the pathogen to the corresponding helper T cell. The presentation is done by integrating it into the cell membrane and displaying it attached to an MHC class II molecule, indicating to other white blood cells that the macrophage is not a pathogen, despite having antigens on its surface. Eventually, the antigen presentation results in the production of antibodies that attach to the antigens of pathogens, making them easier for macrophages to adhere to with their cell membrane and phagocytose.

The term “meninges” as used herein refers to the three layers of tissue that surround the brain and spinal cord.

The term “monocyte” as used herein refers to a type of immune cell that is made in the bone marrow and travels through the blood to tissues in the body where it becomes a macrophage. A monocyte is a type of white blood cell and a type of phagocyte.

The terms “neutrophils” or “polymorphonuclear neutrophils (PMNs)” are used interchangeably to refer to the most abundant type of white blood cells in mammals, which form an essential part of the innate immune system. They form part of the polymorphonuclear cell family (PMNs) together with basophils and eosinophils. Neutrophils are normally found in the blood stream. During the beginning (acute) phase of inflammation, particularly as a result of bacterial infection and some cancers, neutrophils are one of the first-responders of inflammatory cells to migrate toward the site of inflammation. They migrate through the blood vessels, then through interstitial tissue, following chemical signals such as interleukin-8 (IL-8) and C5a in a process called chemotaxis.

The term “normal healthy control subject” as used herein refers to a subject having no symptoms or other evidence of a disease, such as a platelet-dependent disease.

The term “parenteral” as used herein refers to introduction into the body by way of an injection (i.e., administration by injection), including, for example, subcutaneously (i.e., an injection beneath the skin), intramuscularly (i.e., an injection into a muscle), intravenously (i.e., an injection into a vein), intrathecally (i.e., an injection into the space around the spinal cord or under the arachnoid membrane of the brain), intrasternal injection or infusion techniques. A parenterally administered composition is delivered using a needle, e.g., a surgical needle. The term “surgical needle” as used herein, refers to any needle adapted for delivery of fluid (i.e., capable of flow) compositions into a selected anatomical structure. Injectable preparations, such as sterile injectable aqueous or oleaginous suspensions, may be formulated according to the known art using suitable dispersing or wetting agents and suspending agents.

The term “parenchyma” as used herein refers to the tissue of an organ, for example in the brain, that supports its functions and is distinct from supporting and connective tissue.

The term “pellet” as used herein refers to a collection of mass on the bottom and/or side of a container following centrifugation. The term “pellet” as used herein also refers to make or form into pellets.

The term “pharmaceutically acceptable salt” means those salts which are, within the scope of sound medical judgment, suitable for use in contact with the tissues of humans and lower animals without undue toxicity, irritation, allergic response and the like and are commensurate with a reasonable benefit/risk ratio. Examples of pharmaceutically acceptable salts include, but are not limited to, those formed with free amino groups such as those derived from hydrochloric, phosphoric, sulfuric, acetic, oxalic, tartaric acids, and the like, and those formed with free carboxylic groups such as those derived from sodium, potassium, ammonium, calcium, ferric hydroxides, isopropylamine, triethylamine, 2-ethylamino ethanol, histidine, procaine, and the like.

The term “plasma” as used herein refers to the fluid component of the blood in which the particulate material is suspended. The plasma makes up about 55% of the whole blood and contains proteins such as albumins, globulins, and fibrinogen, water, ions, nutrients, and platelets. The plasma does not contain blood cells such as erythrocytes and leukocytes.

The term “platelet” as used herein refers to a cell fragment, lacking a nucleus, that breaks off from a megakaryocyte in the bone marrow and is found in large numbers in the bloodstream. Platelets help initiate blood clotting when blood vessels are injured.

The term “platelet activation” as used herein refers to the process whereby a functionally resting platelet is stimulated to secrete one or more factors involved in thrombus formation or inflammation, or to aggregate. The process of platelet activation involves the expression of activities not shared by functionally resting platelets, including, for example, ATP release, serotonin release, cell surface expression of markers of activated platelets (including, but not limited to, P-selectin and activated GPIIb/IIIa). Alternatively, “platelet activation” as used herein refers to the ability of platelets to aggregate with each other or as the process whereby a platelet gains the expression of one or more of the above-described activities.

The term “platelet-dependent disease” as used herein refers to a disease wherein the patient exhibits an elevated level of an inflammatory chemokine as a result of platelet dysfunction. According to some embodiments, inflammatory chemokines include, but are not limited to, Monocyte Chemotactic Protein-1 (MCP-1/CCL2), Macrophage Inflammatory Protein 1-alpha (MIP-1α/CCL3), Macrophage Inflammatory Protein-1 beta (MIP-1β/CCL4), Regulated upon Activation, Normal T cell Expressed and Secreted (RANTES/CCL5), Platelet Factor-4 (CXCL4/PF4), or a combination thereof.

The term “precipitate,” when referring to centrifugation, refers to the fraction of the composition that is precipitated (or pelleted) during centrifugation to form a cell/cell debris mass.

The term “prevent” as used herein refers to the keeping, hindering or averting of an event, act or action from happening, occurring, or arising.

The term “purification” as used herein refers to the process of isolating or freeing from foreign, extraneous, or objectionable elements.

The term “recombinant” as used herein refers to a substance produced by genetic engineering.

The term “reduced” or “to reduce” as used herein refer to a diminution, a decrease, an attenuation or abatement of the degree, intensity, extent, size, amount, density or number.

The term “similar” is used interchangeably with the terms analogous, comparable, or resembling, meaning having traits or characteristics in common.

The term “solution” as used herein refers to a homogeneous mixture of two or more substances. It is frequently, though not necessarily, a liquid. In a solution, the molecules of the solute (or dissolved substance) are uniformly distributed among those of the solvent.

The terms “subject” or “individual” or “patient” are used interchangeably to refer to a member of an animal species of mammalian origin, including but not limited to, a mouse, a rat, a cat, a goat, sheep, horse, hamster, ferret, platypus, pig, a dog, a guinea pig, a rabbit and a primate, such as, for example, a monkey, ape, or human.

The term “substantially free of” or “essentially free of” are used interchangeably to mean that the blood sample does not contain any detectable amount of a substance, molecule, or cell when analyzed by conventional techniques. For example, the term “substantially free of” or “essentially free of” refers to considerably or significantly free of, or more than about 95% free of, more than about 96% free of, more than 97% free of, more than 98% free of, more than about 99% free of, more that 99.5% free of, more than 99.6% free of, more than 99.7% free of, more that 99.8% free of, or more that 99.9% free of.

The term “a speed sufficient to pellet” as used herein refers to a speed (rpm or g force) of a centrifuge, which can separate and precipitate all detectable amount of substance, molecule, or cell when analyzed by conventional techniques.

The term “supernatant” as used herein refers to the fraction of the composition that is not precipitated (or pelleted) during centrifugation, for example, the fraction of the composition that remains in an aqueous phase in the composition and is substantially free of cells or cell debris.

The term “susceptible” as used herein refers to a member of a population at risk.

The term “symptom” as used herein refers to a phenomenon that arises from and accompanies a particular disease or disorder and serves as an indication of it.

The term “syndrome” as used herein, refers to a pattern of symptoms indicative of some disease or condition.

The term “therapeutic agent” as used herein refers to a drug, molecule, nucleic acid, protein, composition or other substance that provides a therapeutic effect. The term “active” as used herein refers to the ingredient, component or constituent of the compositions of the present invention responsible for the intended therapeutic effect. The terms “therapeutic agent” and “active agent” are used interchangeably. The term “therapeutic component” as used herein refers to a therapeutically effective dosage (i.e., dose and frequency of administration) that eliminates, reduces, or prevents the progression of a particular disease manifestation in a percentage of a population. An example of a commonly used therapeutic component is the ED₅₀ which describes the dose in a particular dosage that is therapeutically effective for a particular disease manifestation in 50% of a population.

The term “therapeutic effect” as used herein refers to a consequence of treatment, the results of which are judged to be desirable and beneficial. A therapeutic effect may include, directly or indirectly, the arrest, reduction, or elimination of a disease manifestation. A therapeutic effect may also include, directly or indirectly, the arrest reduction or elimination of the progression of a disease manifestation.

The term “therapeutically effective amount” or an “amount effective” of one or more of the active agents is an amount that is sufficient to provide the intended benefit of treatment. An effective amount of the active agents that can be employed ranges from generally 0.1 mg/kg body weight and about 50 mg/kg body weight. However, dosage levels are based on a variety of factors, including the type of injury, the age, weight, sex, medical condition of the patient, the severity of the condition, the route of administration, and the particular active agent employed. Thus the dosage regimen may vary widely, but can be determined routinely by a surgeon using standard methods.

The term “treat” or “treating” includes abrogating, substantially inhibiting, slowing or reversing the progression of a disease, condition or disorder, substantially ameliorating clinical or esthetical symptoms of a condition, substantially preventing the appearance of clinical or esthetical symptoms of a disease, condition, or disorder, and protecting from harmful or annoying symptoms. The term “treat” or “treating” as used herein further refers to accomplishing one or more of the following: (a) reducing the severity of the disorder; (b) limiting development of symptoms characteristic of the disorder(s) being treated; (c) limiting worsening of symptoms characteristic of the disorder(s) being treated; (d) limiting recurrence of the disorder(s) in patients that have previously had the disorder(s); and (e) limiting recurrence of symptoms in patients that were previously symptomatic for the disorder(s).

The term “white blood cells” or “WBCs” or “leukocytes” as used herein refers to cells of the immune system that defend the human body against infectious disease and foreign materials. The name “white blood cell” derives from the fact that after centrifugation of a blood sample, the white cells are found in a thin, typically white layer (“buffy coat”) of nucleated cells between the pelleted red blood cells and the blood plasma. The several different types of WBCs, including neutrophils, eosinophils, basophils, lymphocytes, monocytes, macrophages and dendritic cells, often divided into two subgroups, granulocytes or agranulocytes, based on their appearance by light microscopy.

I. Methods for Identifying a Disease or Disorder Treatable with N-Acetylcysteine

According to one aspect, the described invention provides a method for identifying a disease or disorder treatable with N-acetylcysteine or a derivative thereof in a subject, wherein the method comprises:

(a) collecting a whole blood sample from the subject with the disease or disorder, wherein the whole blood sample comprises blood cells and platelets;

(b) separating the blood cells and the platelets from the whole blood sample;

(c) obtaining a purified blood sample, wherein the purified blood sample is substantially free of the blood cells and the platelets;

(d) measuring an amount of at least one marker for platelet dysfunction in the purified blood sample obtained in (c); and

(e) comparing the amount of the at least one marker for platelet dysfunction in the purified blood sample measured in (d) with the amount of the at least one marker for platelet dysfunction in a control blood sample;

wherein the platelets are not activated during steps (a) through (c),

wherein an increased amount of the at least one marker for platelet dysfunction in the purified blood sample compared to the amount of the at least one marker in the control blood sample indicates that the disease or disorder is treatable with N-acetylcysteine.

According to one embodiment of the method, separating step (b) is carried out by centrifugation.

According to another embodiment, separating step (b) is carried out using a fractionation technique, including, but not limited to, flow cytometry, image cytometry, and fluorescence-activated cell sorting (FACS).

Flow cytometry, a technique that may be used for counting and examining cells, allows simultaneous multiparametric analysis of the physical and/or chemical characteristics of each individual cell. Briefly, a beam of light (usually laser light) of a single wavelength is directed onto a hydrodynamically-focused stream of fluid. A number of detectors are aimed at the point where the stream passes through the light beam: one in line with the light beam (Forward Scatter (FSC)), several perpendicular to it (Side Scatter (SSC)), and one or more fluorescence detectors. Each suspended cell (from 0.2 μm to 150 μm) passing through the light beam scatters the light in some way, and fluorescent molecules (naturally occurring or as part of an attached label or dye) may be excited into emitting light at a longer wavelength than the light source. This combination of scattered and fluorescent light is recorded by the detectors. The FSC correlates with the cell volume; SSC depends upon the inner complexity of the cell (i.e., shape of the nucleus, type of cytoplasmic granules, etc.). The data generated by flow cytometers may be plotted as a histogram. The regions on these plots can be separated sequentially based on fluorescence intensity by creating a series of subset extractions (“gates”). Specific gating protocols have been developed for diagnostic and clinical purposes.

Flow cytometry is increasingly used to investigate platelet function against specific antigens expressed on platelets. For platelet investigations there are two main proteins that can be identified; those required for the binding of the platelet to the cell wall and those required for aggregating platelets to each other. Glycoprotein Ib is a surface antigen that is required for binding platelets to the subendothelial matrix via von Willebrand's factor. Glycoprotein IIb/IIIa is one of the main components required for the platelet aggregation reaction. A deficit of platelet function can therefore be identified using monoclonal antibodies raised against either of these proteins.

Fluorescence activated cell sorting (FACS) provides a method of sorting a heterogeneous mixture of cells into two or more containers, a single cell at a time, based upon the specific light scattering and fluorescent characteristics of each cell. Briefly, the cell suspension is entrained in the center of a narrow, rapidly flowing stream of liquid and the flow is arranged such that there is a large separation between cells relative to their diameter. The stream of individual cells passes through a fluorescence detector, and an electrical charge is assigned to each cell (based on the cell's fluorescence) just as the stream is broken into individual drops (usually via vibration) such that there is a low probability of more than one cell per droplet. Each charged droplet (containing an individual cell) may be sorted, via electrostatic deflection, into separate containers.

The surfaces of all cells in the body are coated with specialized protein receptors that selectively can bind or adhere to other signaling molecules. These receptors and the molecules that bind to them are used for communicating with other cells and for carrying out proper cell functions in the body. Each cell type has a certain combination of receptors (or surface markers) on its surface that makes it distinguishable from other kinds of cells. Cells may be fluorescently labeled, i.e., a reactive derivative of a fluorophore may be covalently attached to a cell. The most commonly used labeled molecules are antibodies; their specificity towards certain surface markers on a cell surface allows for more precise detection and monitoring of particular cells. The fluorescence labels that can be used will depend upon the lamp or laser used to excite the fluorochromes and on the detectors available. For example, when a blue argon laser (448 nm) is used, fluorescent labels used may include, but are not limited to, fluorescein isothiocyanate (FITC), Alexa Fluor® 488, green fluorescent protein (GFP), carboxyfluorescein (CFSE), carboxyfluorescein diacetate succinimidyl ester (CFDA-SE), DyLight® 488 (Dyomics), phycoerythrin (PE), propidium iodide (PI), peridinin chlorophyll protein (PerCP), PerCP-Cy™5.5, PE-AlexaFluor 700, PE-Cy™5; PE-Cy™5.5, PE-AlexaFluor® 750 and PE-Cy™7; when a red diode laser (635 nm) is used, fluorescent labels used may include, but are not limited to, allophycocyanin (APC), APC-Cy™7, APC-eFluor® 780, AlexFluor® 700, Cy™5, and Drag-5; when a violet laser is used (405 nm), fluorescent labels may include, but are not limited to, Pacific Orange™, amine aqua, Pacific Blue™, 4′-6-diamidino-2-phenylindole (DAPI), AlexFluor® 405, and eFluor® 450.

Image cytometry is an image-based study or measurement of cells. It differs from conventional microscopic studies of cells in that very large population of cells (typically on the order of 10⁴ to 10⁸ cells) are imaged.

According to another embodiment, separating step (b) is carried out by conventional and confocal microscopy.

According to another embodiment, the at least one marker for platelet dysfunction is an inflammatory chemokine Examples of the inflammatory chemokine include, but are not limited to, Monocyte Chemotactic Protein-1 (MCP-1/CCL2), Macrophage Inflammatory Protein 1-alpha (MIP-1α/CCL3), Macrophage Inflammatory Protein-1 beta (MIP-1β/CCL4), Regulated upon Activation, Normal T cell Expressed and Secreted (RANTES/CCL5), Platelet Factor-4 (CXCL4/PF4), or a combination thereof.

According to another embodiment, the marker for platelet dysfunction is Regulated upon Activation, Normal T cell Expressed and Secreted (RANTES/CCL5).

According to another embodiment, the marker for platelet dysfunction is platelet factor-4 (CXCL4/PF4).

According to another embodiment, measuring step (d) can be carried out by a fluid-based assay, including, but not limited to, enzyme-linked immunosorbent assay (ELISA), bead-based immunoassay (such as, cytometric bead array or Luminex® extracellular assay), mass spectrometry, and nuclear magnetic resonance spectroscopy.

The enzyme-linked immunosorbent assay (ELISA) employs highly-purified capture antibodies that are non-covalently adsorbed (“coated”) onto plastic microwell plates. After washings, the immobilized antibodies capture specifically soluble proteins (e.g., chemokine) present in samples applied to the plate. After washing away unbound material, the captured proteins are detected by biotin-conjugated detection antibodies followed by an enzyme-labeled avidin or streptavidin reporter. Following addition of a chromogenic (color-developing) substrate-containing solution, the level of colored product generated by the bound, enzyme-linked, detection reagents can be measured spectrophotometrically using an ELISA-plate reader at an appropriate optical density.

Cytometric bead array (BD Biosciences, San Jose, Calif.) is a flow cytometry application that allows users to quantify multiple proteins simultaneously. The system employs the broad dynamic range of fluorescence detection offered by flow cytometry and antibody-coated beads to efficiently capture analytes. Each bead in the array has a unique fluorescence intensity so that beads can be mixed and run simultaneously in a single tube. This method significantly reduces sample requirements and time to results in comparison with traditional ELISA and Western blot techniques.

Luminex® extracellular assay (Luminex, Austin, Tex.) combines flow cytometry, micropsheres, lasers, digital signal processing and traditional chemistry. Specifically, the assay utilizes Luminex® color-codes tiny beads, called microspheres, into 100 distinct sets. Each bead set can be coated with a reagent specific to a particular bioassay, allowing the capture and detection of specific analytes from a sample. Within the Luminex® compact analyzer, lasers excite the internal dyes that identify each microsphere particle, and also any reporter dye captured during the assay. Many readings are made on each bead set, further validating the results. In this way, the technique allows multiplexing of up to 100 unique assays within a single sample, both rapidly and precisely.

Mass spectrometry determines the mass of a molecule by measuring the mass-to-charge ratio (m/z) of its ion. Ions are generated by inducing either the loss or gain of a charge from a neutral species. Once formed, ions are electrostatically directed into a mass analyzer where they are separated according to m/z and finally detected. The result of molecular ionization, ion separation, and ion detection is a spectrum that can provide molecular mass and even structural information.

Nuclear magnetic resonance, or NMR, is a phenomenon, which occurs when the nuclei of certain atoms are immersed in a static magnetic field and exposed to a second oscillating magnetic field. Some nuclei experience this phenomenon, and others do not, dependent upon whether they possess a property called spin. Spectroscopy is the study of the interaction of electromagnetic radiation with matter. Nuclear magnetic resonance spectroscopy is the use of the NMR phenomenon to study physical, chemical, and biological properties of matter. NMR spectroscopy is used routinely by chemists to study chemical structure using simple one-dimensional techniques. Two-dimensional techniques are used to determine the structure of more complicated molecules. These techniques are replacing x-ray crystallography for the determination of protein structure. Time domain NMR spectroscopic techniques are used to probe molecular dynamics in solutions. Solid state NMR spectroscopy is used to determine the molecular structure of solids.

According to another embodiment, the disease or disorder is a mucosal disease.

According to another embodiment, the mucosal disease is cystic fibrosis.

According to another embodiment, the disease or disorder is a nervous system disease or disorder, including but not limited to, autism, autism spectrum disorder, and schizophrenia.

### II. Methods for Monitoring Therapeutic Efficacy of a Drug for Treating a Platelet-Dependent Disease.

According to another aspect, the described invention provides a method for monitoring therapeutic efficacy of a drug in the treatment of a platelet-dependent disease in a subject, wherein the method comprises:

(a) collecting a control whole blood sample from the subject prior to administration of a drug for treating the platelet-dependent disease in the subject, wherein the subject has a platelet-dependent disease, and wherein the control whole blood sample comprises blood cells and platelets;

(b) collecting a test whole blood sample from the subject following administration of the drug, wherein the test whole blood sample comprises blood cells and platelets;

(c) separating the blood cells and the platelets from the control whole blood sample of (a) and from the test whole blood sample of (b);

(d) obtaining a purified control blood sample and a purified test blood sample from (c), wherein the purified control blood sample and the purified test blood sample are substantially free of the blood cells and the platelets;

(e) measuring an amount of at least one marker for platelet dysfunction in the purified control blood sample and in the purified test blood sample obtained in (c); and

(f) comparing the amount of the at least one marker for platelet dysfunction in the purified control blood sample with the amount of the at least one marker for platelet dysfunction in the purified test blood sample;

wherein the platelets are not activated during steps (a) through (d);

wherein a decreased amount of the at least one marker for platelet dysfunction in the purified test blood sample compared to the amount of the at least one marker for platelet dysfunction in the purified control blood sample indicates that the drug being administered or having been administered to the subject is effective for treating the platelet-dependent disease in the subject.

According to one embodiment, collecting step (a) is by venipuncture. According to another embodiment, the venipuncture is by an evacuated tube system. According to another embodiment, the venipuncture is by needle and syringe. According to another embodiment, the venipuncture is by a pin-prick puncture. According to some embodiments, the venipuncture is by a neonatal heel prick.

According to another embodiment, the whole blood sample of step (a) is of a volume of about 1 drop to about 20 drops. According to another embodiment, the whole blood sample volume is of about 5 μl. According to another embodiment, the whole blood sample volume is of a volume of about 25 μl. According to another embodiment, the whole blood sample is of a volume of about 50 μl. According to another embodiment, the whole blood sample is of a volume of about 100 μl. According to another embodiment, the whole blood sample is of a volume of about 200 μl. According to another embodiment, the whole blood sample is of a volume of about 300 μl. According to another embodiment, the whole blood sample is of a volume of about 400 μl. According to another embodiment, the whole blood sample is of about 500 μl. According to another embodiment, the whole blood sample is of a volume of about 1 ml. According to another embodiment, the whole blood sample is of a volume of about 5 ml.

According to one embodiment of the method, separating step (c) is carried out by centrifugation.

According to another embodiment, separating step (c) is carried out using a fractionation technique, including, but not limited to, flow cytometry, image cytometry, and fluorescence-activated cell sorting (FACS).

According to another embodiment, the platelet-dependent disease is characterized by an elevated level of inflammatory chemokines, including, but not limited to, Regulated upon Activation, Normal T cell Expressed and Secreted (RANTES/CCL5) and Platelet Factor-4 (CXCL4/PF4).

According to another embodiment, the drug is N-acetylcysteine or a derivative thereof.

According to another embodiment, the marker for platelet dysfunction is an inflammatory chemokine Examples of the inflammatory chemokine include, but are not limited to, Monocyte Chemotactic Protein-1 (MCP-1/CCL2), Macrophage Inflammatory Protein 1-alpha (MIP-1α/CCL3), Macrophage Inflammatory Protein 1-beta (MIP-1β/CCL4), Regulated upon Activation, Normal T cell Expressed and Secreted (RANTES/CCL5), Platelet Factor-4 (CXCL4/PF4), or a combination thereof.

According to another embodiment, the marker for platelet dysfunction is Regulated upon Activation, Normal T cell Expressed and Secreted (RANTES/CCL5).

According to another embodiment, the marker for platelet dysfunction is platelet factor-4 (CXCL4/PF4).

According to another embodiment, the test whole blood sample is obtained on the date of administration, a day after, two days after, three days after, four days after, five days after, six days after, a week after, two weeks after, three weeks after, a month after, two months after, three months after, four months after, five months after, six months after, seven months after, eight months after, nine months after, ten months after, eleven months after, or one year after administration of the drug.

According to another embodiment, measuring step (e) is carried out by a fluid-based assay, including, but not limited to, enzyme-linked immunosorbent assay (ELISA), bead-based assay (such as, cytometric bead array or Luminex-type assay), mass spectrometry, and nuclear magnetic resonance spectroscopy.

Unless defined otherwise, all technical and scientific terms used herein have the same meaning as commonly understood by one of ordinary skill in the art to which this invention belongs. Although any methods and materials similar or equivalent to those described herein also can be used in the practice or testing of the described invention, the preferred methods and materials are now described. All publications mentioned herein are incorporated herein by reference to disclose and describe the methods and/or materials in connection with which the publications are cited.

Where a range of values is provided, it is understood that each intervening value, to the tenth of the unit of the lower limit unless the context clearly dictates otherwise, between the upper and lower limit of that range and any other stated or intervening value in that stated range is encompassed within the invention. The upper and lower limits of these smaller ranges which may independently be included in the smaller ranges is also encompassed within the invention, subject to any specifically excluded limit in the stated range. Where the stated range includes one or both of the limits, ranges excluding either both of those included limits are also included in the invention.

It must be noted that as used herein and in the appended claims, the singular forms “a”, “and”, and “the” include plural references unless the context clearly dictates otherwise. All technical and scientific terms used herein have the same meaning.

The publications discussed herein are provided solely for their disclosure prior to the filing date of the present application. Nothing herein is to be construed as an admission that the described invention is not entitled to antedate such publication by virtue of prior invention. Further, the dates of publication provided may be different from the actual publication dates which may need to be independently confirmed.

The described invention may be embodied in other specific forms without departing from the spirit or essential attributes thereof and, accordingly, reference should be made to the appended claims, rather than to the foregoing specification, as indicating the scope of the invention.

What is claimed is:

1. A method for treating a disease, disorder or condition comprising an inflammatory component that includes platelet dysfunction comprising:
(a) obtaining a whole blood sample from a subject with the disease or disorder, wherein the whole blood sample comprises blood cells and nonactivated platelets;
(b) purifying the blood sample to yield a purified blood sample substantially free of the blood cells and the nonactivated platelets;
(c) measuring an amount of at least one marker for platelet dysfunction in the purified blood sample of (b);
(e) comparing the amount of the marker for platelet dysfunction in the purified blood sample measured in (c) with the amount of the at least one marker for platelet dysfunction in a control blood sample;
wherein an increased amount of the marker for platelet dysfunction in the purified blood sample compared to the amount of the at least one marker in the control blood sample indicates that the subject is susceptible to treatment with the treatment regimen; and
(f) after determining that the subject is susceptible to treatment with the treatment regimen, implementing the treatment regimen comprising administering a composition comprising a therapeutic amount of N-acetylcysteine or a derivative of N-acetylcysteine containing one or more functional groups selected from the group consisting of an aliphatic group, an aromatic group, a heterocyclic radical group, an epoxide group, and an arene oxide group, and a pharmaceutically acceptable carrier, wherein the therapeutic amount is effective to decrease the inflammation due to platelet dysfunction.

2. The method according to claim 1, wherein the at least one marker for platelet dysfunction is an inflammatory chemokine.

3. The method according to claim 2, wherein the inflammatory chemokine is Regulated upon Activation, Normal T-cell Expressed and Secreted (RANTES/CCL-5).

4. The method according to claim 2, wherein the inflammatory chemokine is Platelet Factor-4 (CXCL-4/PF-4).

5. The method according to claim 1, wherein measuring step (c) is carried out by a fluid-based assay.

6. The method according to claim 5, wherein the fluid-based assay comprises an enzyme-linked immunosorbent assay (ELISA), bead-based immunoassay, mass spectrometry, nuclear magnetic resonance spectroscopy, and a combination thereof.

7. The method according to claim 1, wherein the disease or disorder is a mucosal disease.

8. The method according to claim 7 wherein the mucosal disease is cystic fibrosis.

9. The method according to claim 1, wherein the disease or disorder is a nervous system disease.

10. The method according to claim 9, wherein the nervous system disease is autism.

11. The method according to claim 9, wherein the nervous system disease is an autism spectrum disorder.

12. The method according to claim 9, wherein the nervous system disease is schizophrenia.

13. A method for managing a disease comprising an inflammatory component that includes platelet dysfunction comprising
(a)y monitoring therapeutic efficacy of a treatment regimen for treating the disease in a subject, wherein the treatment regimen comprises administering a dose of a pharmaceutical composition comprising a therapeutic amount of N-acetylcysteine and a pharmaceutically acceptable carrier, by:
(1) obtaining a control whole blood sample from the subject prior to initiating the therapeutic regimen and at least one test whole blood sample from the subject after administering the dose of the pharmaceutical composition, wherein each of the control and test whole blood samples comprise blood cells and nonactivated platelets;
(2) purifying the control and test whole blood samples to yield to yield a control purified blood sample and a test purified blood sample wherein each of the control purified blood sample and the test purified blood sample is substantially free of the blood cells and the nonactivated platelets;
(3) measuring an amount of at least one marker for platelet dysfunction in the control purified blood sample and in the test purified blood sample; and
(4) comparing the amount of the marker for platelet dysfunction in the control purified control blood sample with the amount of the marker for platelet dysfunction in the test purified test blood sample;
wherein a decreased amount of the marker for platelet dysfunction in the test purified test blood sample compared to the amount of the marker for platelet dysfunction in the control purified blood sample indicates that the pharmaceutical composition comprising a therapeutic amount of N-acetylcysteine or a derivative of N-acetylcysteine containing one or more functional groups selected from the group consisting of an aliphatic group, an aromatic group, a heterocyclic radical group, an epoxide group, and an arene oxide group and a pharmaceutically acceptable carrier remains effective for treating the platelet dysfunction and
(b) continuing the treatment regimen.

14. The method according to claim 11, wherein the disease comprising platelet dysfunction is characterized by an elevated level of Regulated upon Activation, Normal T-cell Expressed and Secreted (RANTES/CCL-5) or Platelet Factor-4 (CXCL-4/PF-4).

15. The method according to claim 11, wherein the marker for platelet dysfunction is an inflammatory chemokine.

16. The method according to claim 15, wherein the inflammatory chemokine is Regulated upon Activation, Normal T-cell Expressed and Secreted (RANTES/CCL-5).

17. The method according to claim 15, wherein the inflammatory chemokine is Platelet Factor-4 (CXCL-4/PF-4).

18. The method according to claim 11, wherein measuring step (c) is carried out by a fluid-based assay.

19. The method according to claim 18, wherein the fluid-based assay comprises an enzyme-linked immunosorbent assay (ELISA), bead-based assay (such as, cytometric bead array or Luminex-type assay), mass spectrometry, and nuclear magnetic resonance.

20. The method according to claim 11, wherein the platelet-dependent disease is a mucosal disease.

21. The method according to claim 20, wherein the mucosal disease is cystic fibrosis.

22. The method according to claim 11, wherein the platelet-dependent disease is a nervous system disease.

23. The method according to claim 22, wherein the nervous system disease is autism.

24. The method according to claim 22, wherein the nervous system disease is an autism spectrum disorder.

25. The method according to claim 22, wherein the nervous system disease is schizophrenia.
